(19) United States
(12) Patent Application Publication
(10) Pub. No.: US 2004/0018529 A1
(43) Pub. Date: Jan. 29, 2004

(54) Functional cloning of genes encoding proteins/enzymes involved in proteolytic cleavage

(76) Inventors: Yonghong Li, Cupertino, CA (US); Barbara Cordell, Palo Alto, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/447,655

(22) Filed: May 28, 2003

Related U.S. Application Data
(63) Continuation of application No. 09/691,317, filed on Oct. 18, 2000.
(60) Provisional application No. 60/225,664, filed on Aug. 16, 2000.
(60) Provisional application No. 60/160,559, filed on Oct. 20, 1999.

(51) Int. Cl. C12Q 1/68; C12N 15/85

(52) U.S. Cl. 435/6; 435/455

(57) ABSTRACT

This invention concerns methods and means for the functional cloning of genes encoding proteins or enzymes involved in proteolytic cleavage. The invention is based on the use of caspase expression cassettes comprising the coding sequence of a proteolytic cleavage site flanked by sequences encoding two caspase subunits.

### RELATED APPLICATIONS

This application is a continuation of U.S. patent application Ser. No. 09/691,317, filed Oct. 18, 2000, which claims benefit of priority of Provisional Application No. 60/160,559, filed on Oct. 20, 1999, and Provisional Application No. 60/225,564, filed on Aug. 14, 2000, all of which are hereby incorporated by reference in their entirety.

### BACKGROUND OF THE INVENTION

1. Field of the Invention

The present invention concerns methods and means for the functional cloning of genes encoding proteins or enzymes involved in proteolytic cleavage. These proteins or enzymes can be either the components of the enzyme complex or inhibitors of the enzyme. More specifically, the invention concerns the use of caspase expression cassettes comprising the coding sequence of a proteolytic cleavage site flanked by sequences encoding two caspase subunits. The invention also concerns the generation of a conditionally lethal cell line expressing proteolytic cleavage site flanked by the two caspase subunits under control of an inducible promoter. Such cell lines can be used, for example, for selection of genes the expression of which interferes with the enzymatic cleavage based on the survival of cells infected/transfected with a cDNA expression library, after the induced expression of the caspase cassette that would otherwise be processed and activated to induce apoptosis. Similarly, such cell lines can also be used in screening assays to identify molecules that inhibit the protease responsible for the cleavage of the enzymatic cleavage site present. 2. Description of the Related Art

2.1 Caspases

Caspases are a family of cysteine proteases, that are known to participate in the initiation and execution of programmed cell death (apoptosis) (Salvesen and Dixit, Cell 91:443-446 [1997]; Wolf and Green, J. Biol. Chem. 274:20049-20052 [2000]). The first caspase (now referred to as Caspase-1) was originally designated as interleukin-1β-converting enzyme (ICE) (Thomburry et al., Nature 356:768-774 [1992]; Cerretti et al., Science 356:97-100 [1992]). The subsequent members of the caspase family have also been referred to in the literature by different names, such as caspase-2 (also known as Ich-1); caspase-3 (also known as CPP32, yama, and apopain); caspase-4 (also known as TX, Ich-2, and ICE rel-11); caspase-5 (also known as ICE rel-11 and TY), caspase-6 (also known as Mch2); caspase-7 (also known as Mch3, ICE-LAP3, and CHM-1); caspase-8 (also known as MACH, FLICE and Mch5); caspase-9 (also known as ICE-LAP6 and Mch6); and caspase-10 (also known as Mch4). See, Alnemri et al., Cell 87:171 (1996).

Caspases are expressed in cells in an enzymatically inactive form and become activated by proteolytic cleavage in response to an apoptotic stimulus. The inactive pro-enzyme form consists of a large and a small domain (subunit), in addition to an inhibitory N-terminal domain. Caspase activation involves the processing of the pro-enzyme into the large and small subunits, which occurs internally within the molecule. Caspases are activated either by self-aggregation and auto-processing (as in the initiation of apoptosis), or via cleavage by an activated upstream caspase (as in the execution phase of apoptosis). For review, see, for example, Cohen G. M., Biochem. J. 326: 1-16 (1997).

2.2 The role of Proteases in Biological Processes

Proteases are widespread in nature and perform a number of pivotal functions in various biological processes. Proteases have been an attractive therapeutic target due to their involvement in a number of disease processes. The clotting of blood, for example, is mediated by a cascade of proteases including thrombin, factor Xa, factor VIIa etc. Proteases are also involved in tissue destruction that occurs in osteoporosis and arthritis. Several cysteine proteases such as Cathepsin K and Cathepsin L are secreted by osteoclasts and are responsible for bone degradation. A normal bone structure and skeletal integrity is maintained by a careful balance in the activity of osteoblasts, the cells involved in bone formation, and osteoclasts, the cells involved in bone degradation. Excessive secretion of proteases by osteoclasts results in imbalance in the activity of these two cell types, and leads to porous and brittle bones that characterizes osteoporosis. Cathepsin S, a lysosomal cysteine protease present in B-lymphocytes and macrophages, is implicated in several autoimmune diseases such as rheumatoid arthritis, multiple sclerosis, lupus, and asthma due to its critical role in MHC-II receptor processing. Cathepsin B, yet another cysteine protease is commonly overexpressed in cancer cells and may contribute to metastasis. Similarly, urokinase, a serine protease, has been implicated in tumor growth and metastasis.

Proteases also play a critical role in the replication of a number of viruses. For example, Gag and Gag-Pol polyprotein precursors synthesized by the human immunodeficiency virus (HIV) are processed by the viral protease into the structural (matrix and core protein) and non-structural (reverse transcriptase and integrase) viral proteins. The protease action is responsible for the condensation of the core and is characterized as the morphological maturation of virus particles. The hepatitis C virus (HCV) also codes for a protease that is responsible for the proteolytic processing of a single large polyprotein precursor made by the virus. Because of the important role the proteases play in various viral and non-viral diseases, they are often a target of intense research activity for the development of therapeutics. A genetic screen for the isolation and characterization of site-specific proteases, based on the λ phage cI repressor, has been reported (Sices and Kristie, Proc. Natl. Acad. Sci., USA 95: 2828-2833 [1998]). The present invention can be applied to the screening of novel proteases that are important in various diseases. The strategy described herein is also useful for the discovery of novel protease inhibitors. Even a library of chemicals generated by combinatorial chemistry approach can be screened using the strategy presented herein to identify and further develop novel chemical inhibitors of any target protease. Although protease inhibitors have shown great potential for the treatment of AIDS, it is complicated by the rapid emergence of HIV strains that are resistant to protease inhibitors (Moyle and Gazzard, Drugs 51: 701-712 [1996]; Jacobsen et al., J. Infect. Dis. 173: 1379-1387 [1996]). The assay proposed herein can be adapted for the rapid screening of various clinical isolates of HIV for resistance to a panel of protease inhibitors.

A particularly important example of the biological role of proteases is the pathogenesis of Alzheimer's disease, which involves the formation of extracellular amyloid plaques in the cerebral and limbic cortices, predominantly composed of an approximately 4.2 kDa “core” polypeptide, generally referred to as β-amyloid core protein, or, briefly, β-amyloid protein (Aβ). Aβ is produced by proteolytic cleavage of an integral membrane protein, termed the β-amyloid precursor protein (βAPP). There are at least two proteases involved in the generation of Aβ, referred to as β- and γ-secretases (Citron et al., Neuron 17:171-179 [1996]; Seubert et al., Nature 361:260-263 [1993]; Cai et al., Science 259:514-516 [1993]; and Citron et al., Neuron 14:661-670 [1995]). Heretofore, however, the β-secretase has not been definitely identified (reviewed in Evin et al., Amyloid: Int. J. Exp. Clin. Invest. 1: 263-280 [1994]; Hooper et al., Biochem. J. 321: 265-279 [1997]; Selkoe, D. J. Nature 399: A23-A31 [1999]).

The identification, isolation, purification, and characterization of β-secretase would permit chemical modeling of a critical event in the pathology of Alzheimer's disease and would allow the screening of compounds to determine their ability to inhibit β-secretase activity. For these reasons, it would be desirable to isolate, clone and characterize the enzyme referred to as β-secretase. It would be also desirable to develop a method for the functional cloning of the genes encoding proteins or enzymes involved in the proteolytic cleavage of β-APP. It would further be desirable to identify inhibitors of the β-APP processing leading to Aβ release.

2.3 Membrane Protein Secretases or Sheddases

A group of proteases that specifically deserves mention with regard to the utility of the caspase cassettes and related methods described herein is membrane protein secretases also referred to collectively as ‘sheddases’. The group includes enzymes which cleave membrane integral proteins, of type I as well as type II topology, in order to generate circulating, soluble form of the same protein (reviewed in Hooper et al., Biochem. J. 321: 265-279 [1997]). The cleavage usually occurs close to the extracellular face of the membrane, releasing physiologically active secretory protein. The examples of secretory proteins released from their precursor membrane proteins by the action of secretases include cell adhesion molecules (ICAM-3, VCAM-1, L-selectin), leucocyte antigens (class I MHC, IL-2 receptor, CD44, CD43), ligands (Fas, TNF-α, TGF-β, CD40, CSF-1), receptors (TNFR-I, TNFR-II, CSF-1 receptor, NGF receptor, TGF-β receptor, PDGF receptor, IL-1 receptor, IL-6 receptor) etc. Since these proteins are involved in various pathophysiological processes such as neurodegeneration, apoptosis, oncogenesis and inflammation, the isolation and characterization of secretases involved in theses processes and their inhibitors, by the method detailed herein, could provide novel therapeutic targets.

In addition to the need for the identification of β-secretase and its inhibitors, the identification, isolation, and characterization of novel molecules having unique activities, such as enzymatic activities, is generally useful. For example, novel enzymes can be used to catalyze reactions of a type associated with their class. Novel proteases can be used to cleave proteins for a variety of purposes, and the availability of new proteases provides unique capabilities.

### SUMMARY OF THE INVENTION

Caspases play a major role in the transduction of the apoptotic signal and execution of apoptosis in mammalian cells. As discussed before, caspases are proteases that exist as pro-enzymes, activated by cleavage into a large and a small subunit, occurring after specific aspartic acid residues within the pro-enzyme sequence. The present invention is based on the premise that if the native enzyme recognition (cleavage) site of a caspase is replaced by a site that is recognized and cleaved by another protein or enzyme, the processing, and hence the activation, of the caspase units will be subject to the availability of the protein/enzyme of interest. Accordingly, introduction of an expression vector containing such a caspase construct (“cassette”) into cells might or might not result in apoptosis, depending on whether or not the cells express the protein/enzyme of interest, and whether or not the cells are susceptible to the caspase activity. If a particular cell does contain the protein/enzyme of interest, and undergoes apoptosis upon caspase activation, the presence of the protein/enzyme can then be functionally coupled to cell death. This coupling can be of various uses. For example, stable cell lines conditionally expressing the caspase cassette can be generated with the aid of an inducible expression system. When the expression of the caspase cassette is artificially induced, these cells would normally undergo apoptosis but will survive if the caspase cassette cannot be processed. Processing does not occur, for example, if the expression of the protein/enzyme of interest is knocked out or down-regulated by an anti-sense cDNA, or if the expression and/or activity of the protein/enzyme is inhibited by its inhibitors or dominant negative mutants. Therefore, these cells can be used to genetically screen for suppressors of the expression or activity of the protein/enzyme.

In one aspect, the invention concerns a fusion polypeptide comprising a first and a second caspase subunit, separated by a cleavage site not associated in nature with at least one of the caspase subunits, where, upon release, the first and second caspase subunits are capable of participating in the induction of apoptosis. The fusion polypeptide preferably contains subunits from a native eukaryotic caspase, more preferably from a native human caspase, although caspase subunits not occurring in nature may also be used, provided that, upon release, they retain the ability to participate in the induction of apoptosis. Preferably, the native human caspase subunits are from one or more of the following caspases: caspase-2, caspase-3, caspase-6, caspase-7, caspase-8, caspase-9 and caspase-10. Even more preferably, the large and small subunits of native human caspase-3 are used in the fusion polypeptides of the present invention. In a particularly preferred embodiment, the native enzyme recognition site of a caspase, e.g. caspase-3, is replaced by the cleavage site of a secretase, such as α-, β- or γ-secretase, preferably β-secretase.

In another aspect, the invention concerns a polynucleotide comprising a sequence encoding a fusion polypeptide having a first and a second caspase subunit, separated by a cleavage site not associated in nature with at least one of the caspase subunits, where, upon release, the first and second caspase subunits are capable of participating in the induction of apoptosis. The polynucleotide preferably comprises sequence encoding for any of the preferred fusion polypeptides described above.

In yet another aspect, the invention concerns a vector comprising, and capable of expressing a polynucleotide as hereinabove defined, and recombinant host cells transformed with such vector. Preferably, the recombinant host cell is an eukaryotic cell susceptible to caspase activity. In a particular, although not critical, embodiment, the recombinant host cell does not endogenously express the caspase from which the caspase subunit is derived.

In a further aspect, the invention concerns a method for producing a polypeptide by expressing, in a suitable recombinant host cell a polynucleotide comprising a sequence encoding a fusion polypeptide having a first and a second caspase subunit, separated by a cleavage site not associated in nature with at least one of the caspase subunits, where, upon release, the first and second caspase subunits are capable of participating in the induction of apoptosis, and isolating the polypeptide from the cell or cell culture.

In a still further aspect, the invention concerns a method of identifying nucleic acid that encodes or acts as an inhibitor of a target polypeptide involved in proteolytic cleavage, comprising:

(a) transfecting/infecting, with a cDNA library, cells that are susceptible to caspase activity and express a nucleic acid encoding the target polypeptide and a polynucleotide encoding a fusion polypeptide comprising a first and a second caspase subunit, separated by a cleavage site capable of cleavage by said target polypeptide;

(b) selecting surviving cells; and

(c) identifying the inhibitory nucleic acid in the surviving cells.

In a specific embodiment, the inhibitor is an antisense transcript of the DNA encoding the target polypeptide. In another embodiment, the inhibitor is a trans-dominant derivative of the target polypeptide. In yet another embodiment, the inhibitor negatively regulates the activity of the target polypeptide. In a further embodiment, the inhibitor inhibits the expression of the target polypeptide. The library preferably is a cDNA expression library, more preferably a retroviral cDNA expression library. In performing this method, the caspase expression cassette is under control of an inducible promoter.

In another aspect, the invention concerns a method of cloning a gene encoding a target polypeptide involved in proteolytic cleavage comprising

(a) transfecting/infecting a stable cell line susceptible to caspase activity and expressing a gene encoding the polypeptide of interest, with an expression cassette, containing a fusion polypeptide comprising a first and a second caspase subunit, separated by a cleavage site capable of cleavage by the polypeptide of interest, under control of an uninduced inducible promoter;

(b) transfecting/infecting the cell line with a cDNA expression library;

(c) inducing the expression of the fusion polypeptide;

(d) isolating the cell clones that survive induction; and

(e) isolating and identifying the cDNA rescuing the cell clones from death.

In a specific embodiment, the caspase cassette corresponds to the subunits of a native human caspase, such as caspase-3, and the cell line shows no endogenous expression of the corresponding caspase, e.g. caspase-3. The inducible promoter may, for example, be a modified CMV promoter that is under the control of a tetracycline-inducible system, and, if the polypeptide of interest is β-secretase, a suitable cell line is MCF-7 or 293 HEK.

In yet another aspect, the invention concerns a method of identifying a mutant cell line deficient in an enzyme of interest, comprising

(a) transfecting/infecting cells susceptible to caspase activity and normally expressing the gene encoding the enzyme of interest, with an expression cassette, containing a fusion polypeptide comprising a first and a second caspase subunit, separated by a cleavage site capable of cleavage by the enzyme of interest;

(b) monitoring cell death; and

(c) identifying surviving cells.

The death is typically monitored in comparison with cells co-transfected with nucleic acid encoding the first and second caspase subunits present in the caspase expression cassette.

In a different aspect, the invention concerns a method for the diagnosis of the presence of a tumor characterized by the selective expression of a gene encoding a polypeptide involved in proteolytic cleavage comprising:

(a) transfecting cells obtained from an individual to be diagnosed with an expression cassette, containing a fusion polypeptide comprising a first and a second caspase subunit, separated by a cleavage site capable of cleavage by the polypeptide involved in proteolytic cleavage, and

(b) monitoring cell death compared with corresponding cells obtained from a healthy individual. Death of cells obtained from the patient to be diagnosed is indicative of the presence of tumor.

The invention further concerns a method of suppressing proliferation of a tumor cell characterized by the selective expression of a polypeptide involved in proteolytic cleavage, comprising introducing into the tumor cell an expression cassette which comprises a polynucleotide that encodes a cleavage site recognized and cleaved by the polypeptide, flanked by two caspase subunits. Following the introduction of the caspase expression cassette, the polypeptide selectively expressed in the tumor cell recognizes the cleavage site, and releases the caspase subunits which, in turn, suppress the proliferation of the tumor cell.

In a particular embodiment, the caspase expression cassette is operably linked to a SV40 early region promoter, a retroviral long-terminal repeat, a cytomegalovirus promoter, a β-actin promoter, a glucocorticoid-inducible promoter, or a herpes simplex virus thymidine kinase promoter. The expression vector may be introduced into the tumor cells, for example by viral infection, liposome-mediated transfection, polybrene-mediated transfection or CaPO₄-mediated transfection, and the introduction may take place in vitro or in vivo.

In a different aspect, the invention concerns a method for identifying a molecule that acts as an inhibitor of a target polypeptide, comprising

(a) contacting a fusion polypeptide which comprises a cleavage site normally recognized and cleaved by such polypeptide, flanked by two casepase subunits, with a plurality of molecules under conditions conducive to cleavage; and

(b) identifying a molecule within the plurality of molecules that inhibits cleavage.

In a further aspect, the invention concerns a cell-based method for identifying a molecule that acts as an inhibitor of a target polypeptide, comprising

(a) incubating a cell transfected/infected with an expression cassette encoding a fusion polypeptide comprising a cleavage site normally recognized and cleaved by such target polypeptide, flanked by two caspase subunits; and

(b) monitoring cell death upon induction of the expression of said expression cassette.

This cell-based approach is particularly suitable for screening for small molecule inhibitors of a target polypeptide.

In a still further aspect, the invention concerns a method of screening for an agent that acts as an inhibitor of a target polypeptide, comprising

(a) incubating a mixture of a candidate agent and a fusion polypeptide which comprises a cleavage site normally recognized and cleaved by such target polypeptide, flanked by two caspase subunits, under conditions whereby, but for the presence of the candidate agent, the target polypeptide cleaves the fusion polypeptide; and

(b) monitoring the cleavage.

The invention also concerns a method for identifying a sequence motif specifically recognized by a protease, comprising

(a) expressing an expression cassette comprising the coding sequence of a candidate sequence motif to be cleaved by such protease, flanked by two caspase subunits, in an eukaryotic host cell, in the presence of the protease;

(b) monitoring cell death; and, if necessary,

(c) repeating steps (a) and (b) with one or more modified candidate sequence motifs until maximum cell death occurs.

In yet another aspect, the invention concerns a method of screening a clinical isolate of HIV for resistance to an anti-HIV protease inhibitor, comprising

(a) incubating the isolate with a cell naturally susceptible to HIV infection, transfected to express a fusion polypeptide comprising a first and second caspase subunit, separated by a cleavage site capable of cleavage by an HIV protease, under control of an uninduced inducible promoter, with or without an inhibitor of HIV protease;

(b) inducing the expression of the fusion polypeptide; and

(c) monitoring cell death.

The cells that are CD4+, such as CD4+T cells or cells of monocyte/macrophage lineage, are suitable for the assay. As a positive control, steps (b) to (c) may be repeated using a strain of HIV containing wild type protease that is susceptible to an anti-HIV protease inhibitors used in AIDS treatment. The HIV isolates that are resistant to a protease inhibitor will kill cells irrespective of the presence or absence of the protease inhibitor, whereas the HIV isolates that are susceptible to a protease inhibitor will kill cells only in the absence of the protease inhibitor.

### BRIEF DESCRIPTION OF THE DRAWINGS

In the following Figures of deduced amino acid sequences, the sequences identified by bold face and underlining represent the proteolytic cleavage sites.

FIG. 1 shows the nucleotide sequence of human caspase-1 (SEQ ID NO: 1).

FIG. 2 shows the deduced amino acid sequence of human caspase-1 (SEQ ID NO: 2).

FIG. 3 shows the nucleotide sequence of human caspase-2 (SEQ ID NO: 3).

FIG. 4 shows the deduced amino acid sequence of human caspase-2 (SEQ ID NO: 4).

FIG. 5 shows the nucleotide sequence of human caspase-3 (SEQ ID NO: 5).

FIG. 6 shows the deduced amino acid sequence of human caspase-3 (SEQ ID NO: 6).

FIG. 7 shows the nucleotide sequence of human caspase-4 (SEQ ID NO: 7).

FIG. 8 shows the deduced amino acid sequence of human caspase-4 (SEQ ID NO: 8).

FIG. 9 shows the nucleotide sequence of human caspase-5 (SEQ ID NO: 9).

FIG. 10 shows the deduced amino acid sequence of human caspase-5 (SEQ ID NO: 10).

FIG. 11 shows the nucleotide sequence of human caspase-6 (SEQ ID NO: 11).

FIG. 12 shows the deduced amino acid sequence of human caspase-6 (SEQ ID NO: 12).

FIG. 13 shows the nucleotide sequence of human caspase-7 (SEQ ID NO: 13).

FIG. 14 shows the deduced amino acid sequence of human caspase-7 (SEQ ID NO: 14).

FIG. 15 shows the nucleotide sequence of human caspase-8 (SEQ ID NO: 15).

FIG. 16 shows the deduced amino acid sequence of human caspase-8 (SEQ ID NO: 16).

FIG. 17 shows the nucleotide sequence of human caspase-9 (SEQ ID NO: 17).

FIG. 18 shows the deduced amino acid sequence of human caspase-9 (SEQ ID NO: 18).

FIG. 19 shows the nucleotide sequence of human caspase-10 (SEQ ID NO: 19).

FIG. 20 shows the deduced amino acid sequence of human caspase-10 (SEQ ID NO: 20).

FIG. 21 is a schematic diagram of a chimeric caspase expression construct including a known enzyme cleavage site.

FIG. 22 illustrates the determination of caspase-induced death of cells (Y) expressing enzyme X, transfected with a chimeric caspase expression cassette containing the coding sequence for the recognition site of enzyme X.

FIG. 23 illustrates the generation of an enzyme X-dependent lethal cell line (Y2).

FIG. 24 is a schematic diagram illustrating the cloning of genes the expression of which blocks the enzyme X-dependent cell death.

FIG. 25 is a schematic diagram of the overlapping PCR.

FIG. 26 shows nucleotide sequence of genetically engineered caspase chimera cassette (SEQ ID NO. 23).

FIG. 27 shows deduced amino acid sequence of genetically engineered caspase chimera cassette (SEQ ID NO. 24). β-secretase cleavage sequence that replaces the naturally occurring caspase cleavage site is underlined. The sequences upstream and downstream of β-secretase cleavage sequence correspond to large subunit and small subunit of caspase-3 respectively.

FIG. 28 The modified caspase-3 induces apoptosis of 293T cells. (A) Schematic representation of the modified caspase-3 containing β-secretase recognition (cleavage) site. p17 and p12 are the large and small subunits of caspase-3, respectively. The APP sequence surrounding the β-secretase cleavage site is given (SEQ ID NO: 29), and the statin peptide inhibitor of β-secretase is boxed. (B) Cell morphology after transfection of various constructs and indicated treatment. 293T cells were either mock-transfected or transfected with indicated plasmids and photographed 24 h after transfection. Note that apoptotic cells appeared bright and rounded. The caspase inhibitor Z-VAD.fmk was added to the culture medium at a final concentration of 10 μM immediately after the 5-h incubation period for the transfection. (C) Morphology of 293T cells transfected with indicated plasmids and observed under an inverted fluorescent (with dim normal light) microscope 24 h after transfection. Note the co-appearance of apoptotic cells and green cells. (D) A representative counting of apoptotic cells. Transfected or green cells showing apoptotic and non-apoptotic features were counted from a randomly chosen field, and the percentage of apoptotic cells were determined based on green cells showing apoptotic signs and the total green cells. Similar results were obtained in 3 independent experiments. (E) A time course of caspase-3 activity following transfection of pCBC1. 293T cells were mock-transfected or transfected with pCBC1 plasmid, collected at various times after transfection, and processed for the caspase-3 assay. The results shown are the mean±SD of a typical experiment with 3 determinations. They are relative to the mock-transfected at 0 h and were normalized by the protein content. Similar results were obtained in 3 independent experiments.

FIG. 29. Correlation between the modified caspase-3 induced apoptosis and the susceptibility of the included APP sequence by β-secretase. (A) APP sequence of various pCBC1 mutants and the relative cleavage of the corresponding APP sequence by β-secretase. The cleavage of APP variants by β-secretase as shown is relative to the wild-type APP. *APP sequence incorporated into the modified caspase-3. **Relative cleavage by β-secretase of the full-length APP sequence containing the indicated mutations, compared with the wild type APP sequence (set at 1.0). The data are derived from Citron et al. (1995) and the studies outlined herein. (B) Apoptosis assay of 293T cells following transfection. Cells were transfected with the indicated plasmids. Twenty-four hour later, 3 fields were randomly chosen, photographed and counted for apoptotic cells. (C) Apoptotic ladder assay. 293T cells were transfected with indicated plasmids, collected 24 h after transfection, and genomic DNA isolated. The samples were then examined for fragmentation using Clontech's apoptotic ladder assay kit. (D) Expression of the caspase-3 by various constructs in 293T cells. Cells were transfected with the plasmids and harvested 24 h later. Equal amounts of total proteins were then subjected to electrophoresis, followed by Western blot assay using an anti-caspase-3 antibody.

FIG. 30. In vivo β-site APP cleavage inhibitors BACE2 and BACE2(D110A) prevent processing and activity of the modified caspase-3 and cell death. (A) BACE2 inhibits the β-secretase cleavage in 293T cells. 293T cells were transfected with indicated plasmids for 48 h and the medium was assayed for β-NTF by ELISA. (B) Effect of BACE2 and BACE2(D110A) (noted as BACE2*) on pCBC1-induced apoptosis of 293T cells. (C) Effect of BACE2 and BACE2(D110A) on the processing of the modified caspase-3 expressed in 293T cells. Cells were transfected with indicated plasmids and cell lysates were subjected to Western blot analysis using an anti-caspase-3 antibody. Equal amount of proteins, as determined by BCA assay, was loaded to each lane. The two panels shown were from the same blot but with different exposure time when the blot was processed by the ECL method. The top panel, from short exposure, shows the full-length endogenous and the modified caspase-3. The bottom panel, from long exposure, shows the small subunit, 12 kDa, of the modified caspase-3. (D) Effect of BACE2 and BACE2(D110A) on caspase-3 activity following co-transfection with pCBC1 into 293T cells. Caspase-3 activity was normalized by the protein content and is presented as relative to the mock control.

### DETAILED DESCRIPTION OF THE PREFERRED EMBODIMENT

5.1 Definitions

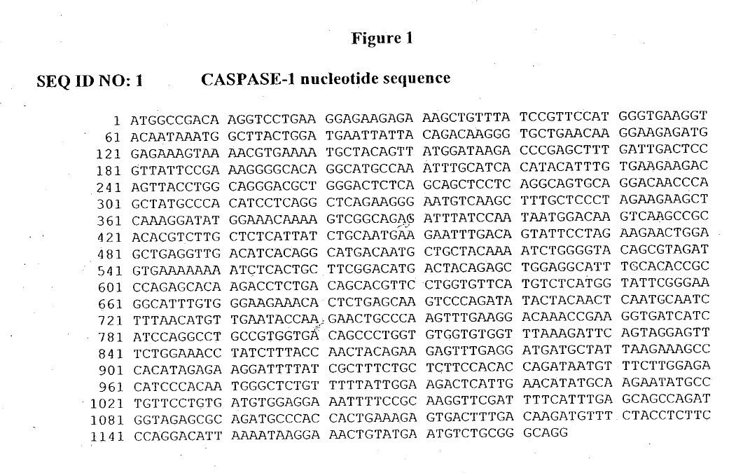

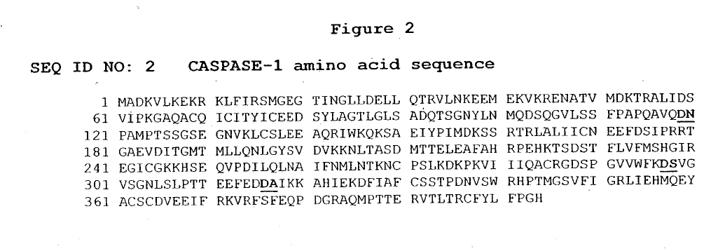

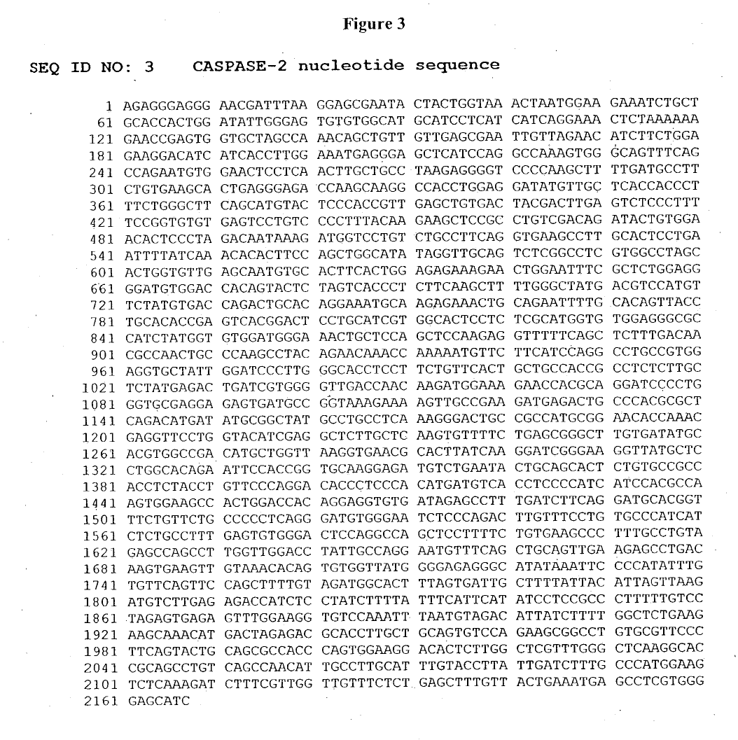

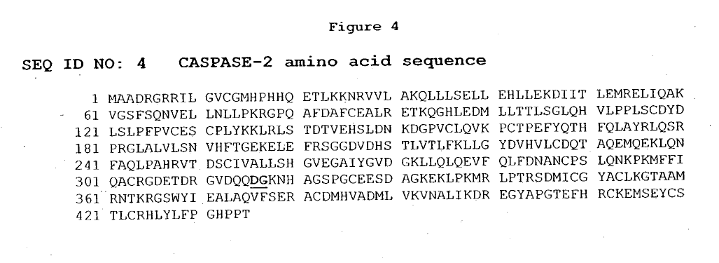

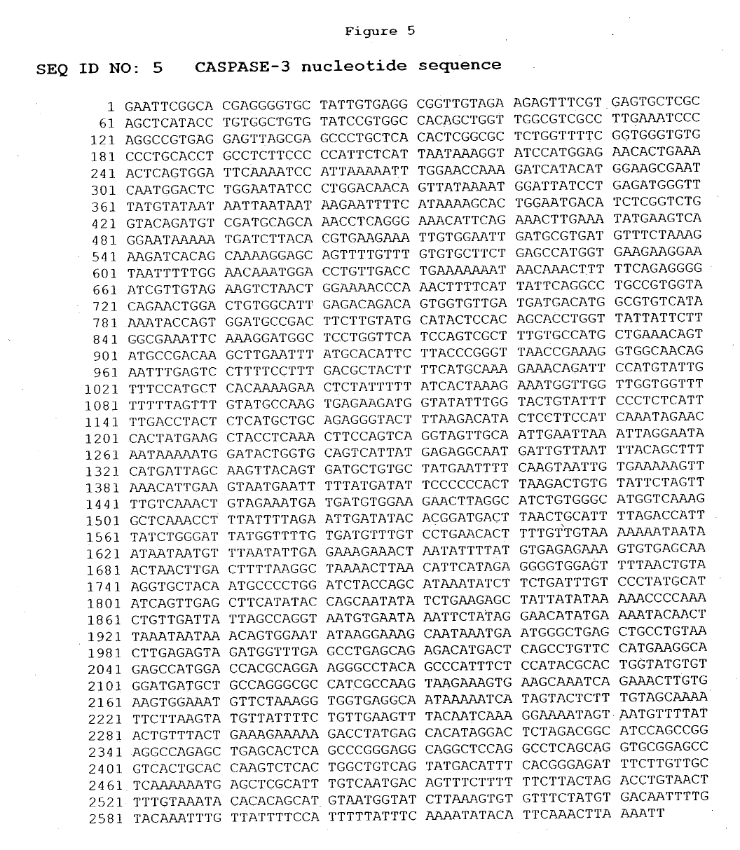

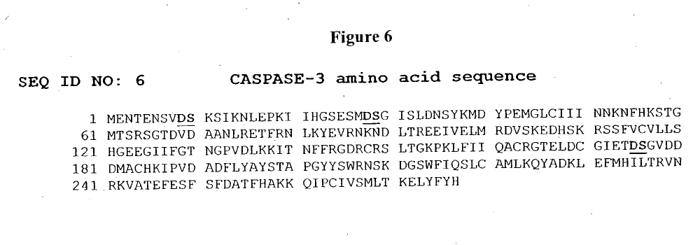

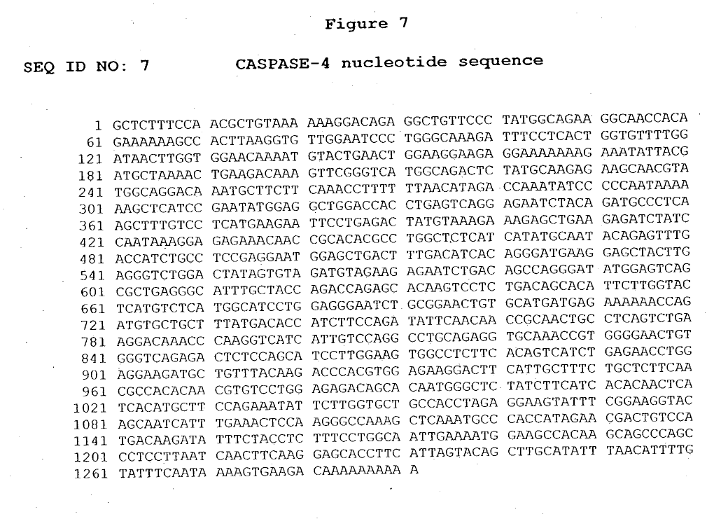

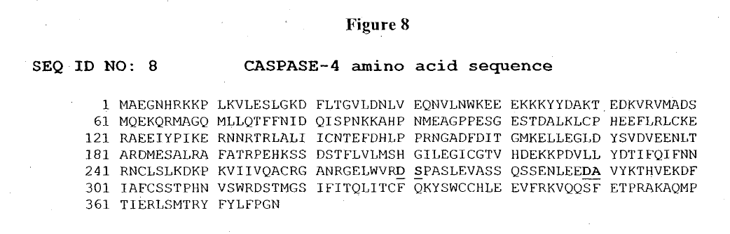

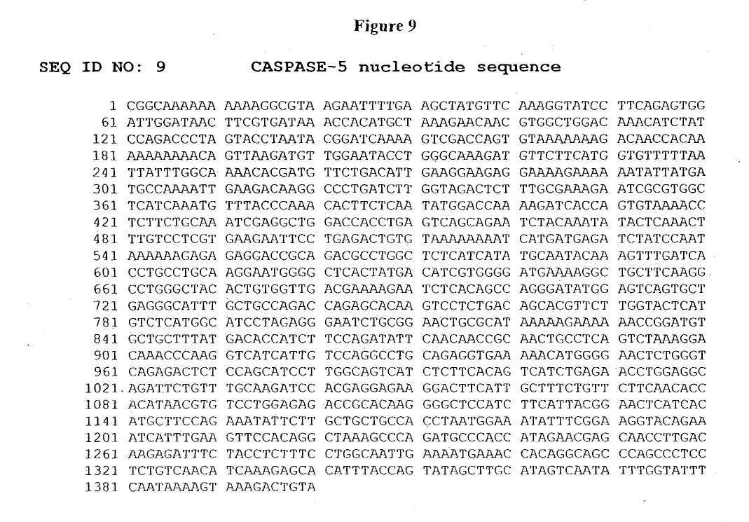

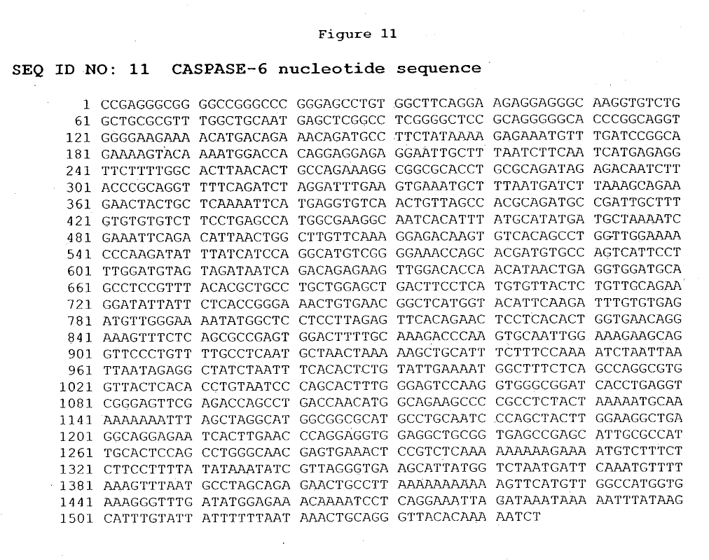

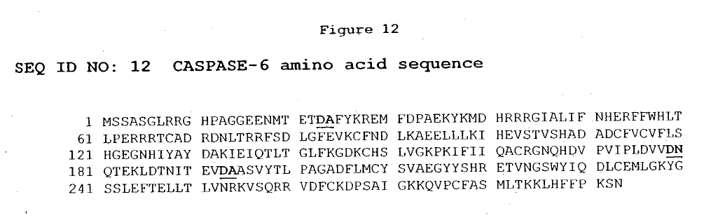

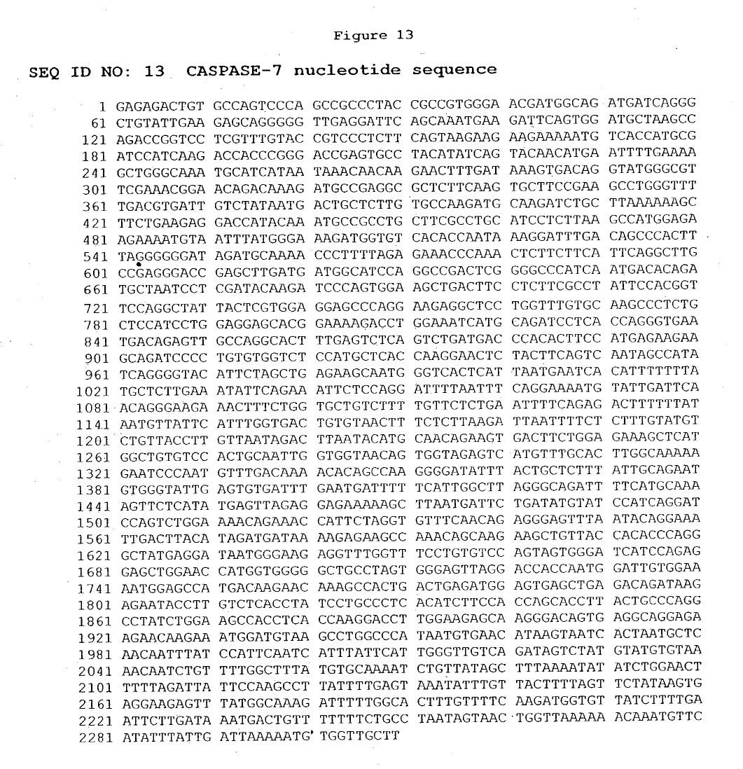

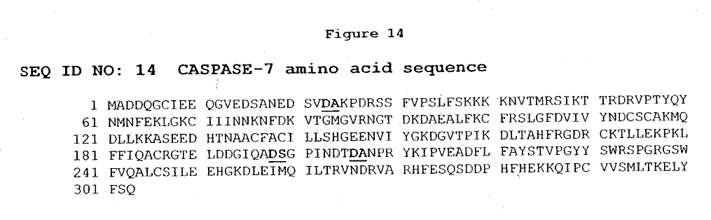

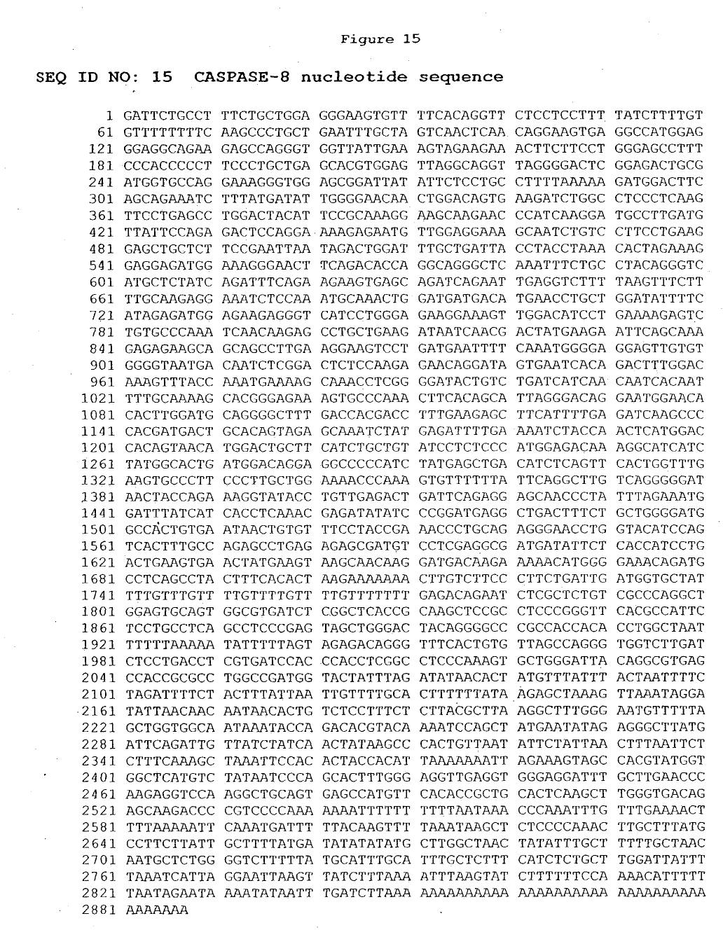

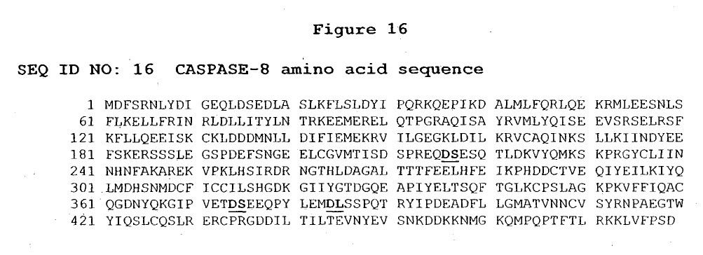

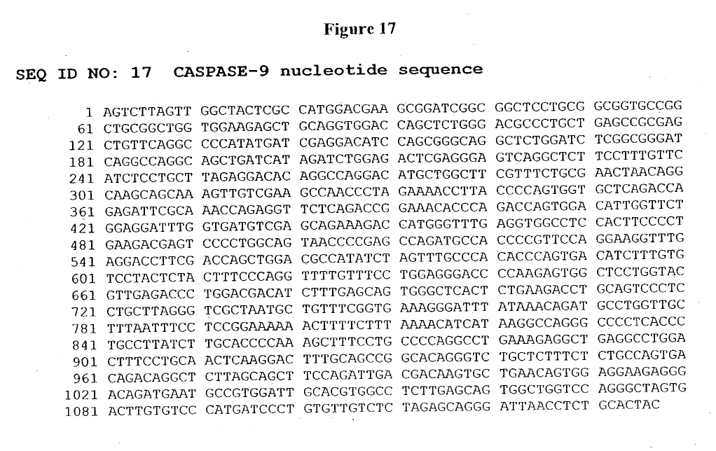

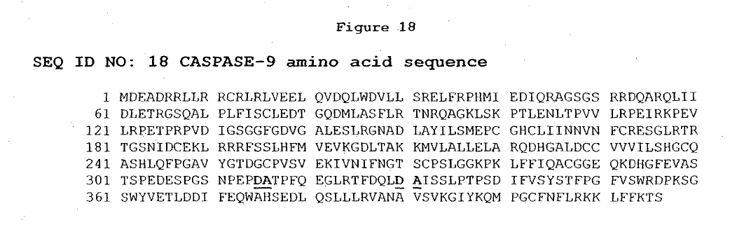

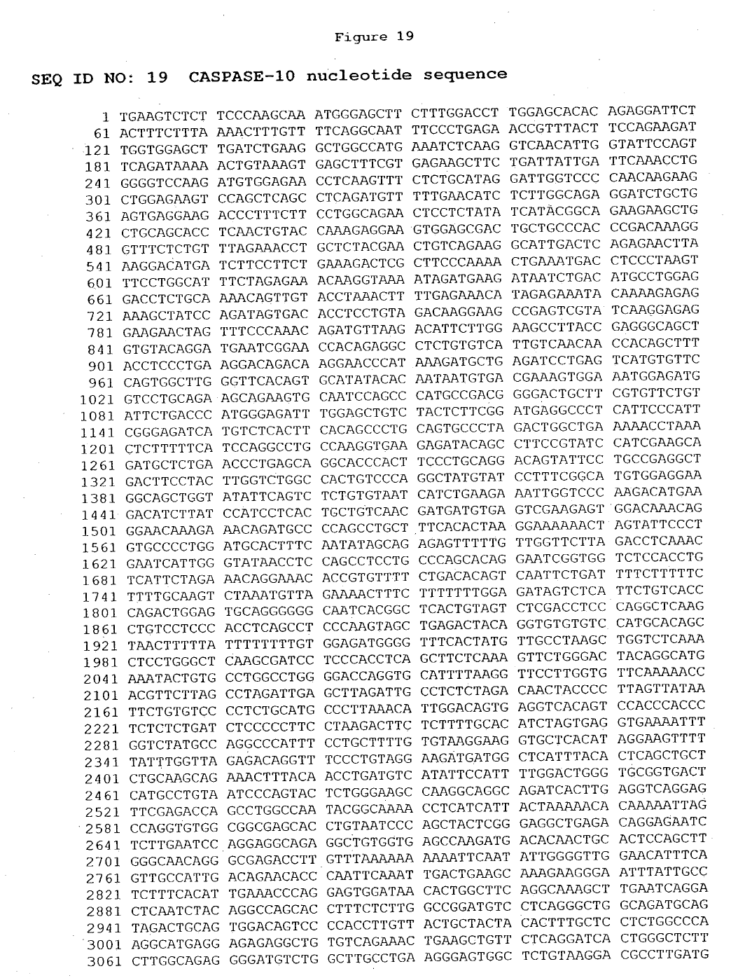

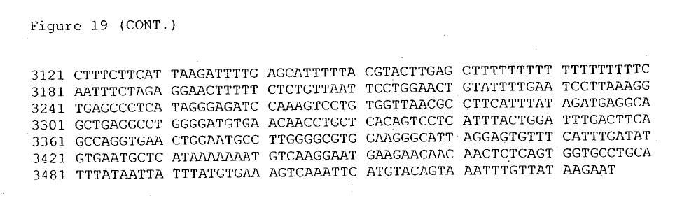

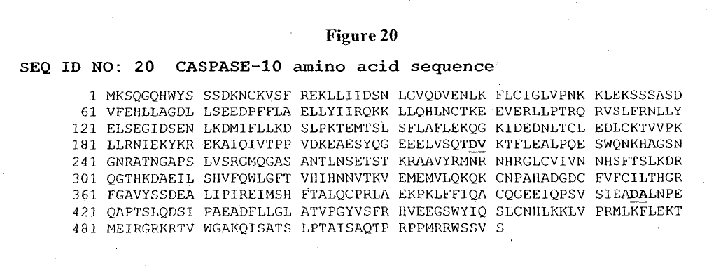

The term “caspase” is used to refer to all members of the caspase family (also known as the ICE family of cysteine proteases), known in the art or hereinafter identified, and their functional derivatives, provided that they are involved in the initiation and/or execution of programmed cell death (apoptosis). The term specifically includes native human caspases 1-10 (FIGS. 1-20, SEQ ID NOS: 1-20), including allelic variants, and their non-human mammalian homologues.

Naturally occurring (native) caspases are synthesized as catalytically inactive zymogenic prenzymes, that are generally activated by cleavages after specific internal aspartic acid (Asp) residues present in interdomain linkers, yielding a large and a small subunit, and are able to cleave their substrates after Asp residues. As a result, certain mature active caspases can process and activate their own (autocatalytic cleavage) and other inactive caspase zymogens. Release of the caspase subunits triggers apoptosis. The term “caspase subunit” is used to refer to subunits of native caspases, as hereinabove defined, generated by internal cleavage of the zymogenic caspase pro-enzymes, which, upon release, induce (initiate and/or execute) apoptosis, and their functional derivatives. The term specifically includes subunits of native human caspases 1-10, including allelic variants, and their non-human eukaryotic homologues.

A “functional derivative” of a native polypeptide is a compound having a qualitative biological activity in common with the native polypeptide. For the purpose of the present invention, a “functional derivative” of a native caspase is defined by its ability to be cleaved into active subunits that participate in the initiation and/or execution of apoptotic cell death. Similarly, a “functional derivative” of a native caspase subunit is a molecule which, together with another caspase subunit (native or modified), participates in the initiation and/or execution of apoptotic cell death. Functional derivatives include, but are not limited to, fragments of native polypeptides from any animal species (including mammals, such as humans), and derivatives of native (human and non-human) polypeptides and their fragments, provided that they retain the ability to participate in the initiation and/or execution of apoptotic cell death.

“Fragments” comprise regions within the sequence of a native caspase polypeptide or polypeptide subunit.

The term “derivative” is used to define amino acid sequence variants, and covalent modifications of a native polypeptide, such as a native caspase of caspase subunit.

The term “amino acid sequence variant” refers to molecules with some differences in their amino acid sequences as compared to a reference (e.g. a native sequence) polypeptide. The amino acid alterations may be substitutions, insertions, deletions, or any desired combinations of such changes in a native (or other reference) amino acid sequence.

Substitutional variants have at least one amino acid residue in a native sequence removed and a different amino acid inserted in its place at the same position. The substitution may be single, where only one amino acid in the molecule has been substituted, or they may be multiple, where two or more amino acids have been substituted in the same molecule.

Insertional variants are those with one or more amino acids inserted immediately adjacent to an amino acid at a particular position in a native amino acid sequence. Immediately adjacent to an amino acid means connected to either the α-carboxy or the α-amino functional group of the amino acid.

Deletional variants are those with one or more amino acids in the native amio acid sequence removed. Deletional variants include N- and C-terminal deletions as well as internal deletions within a native sequence molecule.

Covalent derivatives include modifications of a native polypeptide or a fragment thereof with an organic proteinaceous or non-proteinaceous derivatizing agent, and post-translational modifications. For post-translational modifications see, e.g. T. E. Creighton, Proteins: Structure and Molecular Properties, W. E. Freeman & Co., San Francisco, pp. 79-86 [1983]).

The amino acid sequence variants of the caspase subunits within the constructs of the present invention preferably show at least about 80%, more preferably at least about 90%, most preferably at least about 95% sequence identity with the corresponding sequence of a native caspase subunit. Alternatively, they are encoded by nucleic acid that hybridizes under stringent conditions to the complement of a nucleic acid encoding a native caspase subunit.

“Sequence identity” is defined as the percentage of amino acid residues in a candidate sequence that are identical with the amino acid residues in a native polypeptide sequence, after aligning the sequences and introducing gaps, if necessary, to achieve the maximum percent sequence identity, and not considering any conservative substitutions as part of the sequence identity. The % sequence identity values are generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to −1.

“Stringent” hybridization conditions are sequence dependent and will be different with different environmental parameters (e.g., salt concentrations, and presence of organics). Generally, stringent conditions are selected to be about 5° C. to 20° C. lower than the thermal melting point (Tₘ) for the specific nucleic acid sequence at a defined ionic strength and pH. Preferably, stringent conditions are about 5° C. to 10° C. lower than the thermal melting point for a specific nucleic acid bound to a complementary nucleic acid. The Tₘ is the temperature (under defined ionic strength and pH) at which 50% of a nucleic acid (e.g., tag nucleic acid) hybridizes to a perfectly matched probe. “Stringent” wash conditions are ordinarily determined empirically for hybridization of each set of tags to a corresponding probe array. The arrays are first hybridized (typically under stringent hybridization conditions) and then washed with buffers containing successively lower concentrations of salts, or higher concentrations of detergents, or at increasing temperatures until the signal to noise ratio for specific to non-specific hybridization is high enough to facilitate detection of specific hybridization. Stringent temperature conditions will usually include temperatures in excess of about 30° C., more usually in excess of about 37° C., and occasionally in excess of about 45° C. Stringent salt conditions will ordinarily be less than about 1000 mM, usually less than about 500 mM, more usually less than about 400 mM, typically less than about 300 mM, preferably less than about 200 mM, and more preferably less than about 150 mM. However, the combination of parameters is more important than the measure of any single parameter. See, e.g., Wetmur et al., J. Mol. Biol. 31:349-70 (1966), and Wetmur, Critical Reviews in Biochemistry and Molecular Biology 26(34):227-59 (1991). In a preferred embodiment, “stringent conditions” or “high stringency conditions,” as defined herein, may be hybridization in 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

The terms “induction of apoptosis” and “induction of (programmed) cell death” are used interchangeably and refer to an activity that results in programmed (apoptotic) cell death, regardless of the underlying mechanism. The terms specifically include the initiation and/or execution of apoptosis. “Participation” in the induction of apoptosis means that the molecule, alone or in combination with other components of the apoptotic pathway, plays a role in the initiation and/or execution of apoptosis.

The term “inhibitor” is used in the broadest sense and refers to a suppressor of the expression and/or activity of a polypeptide of interest. The inhibitor may completely block or, at least, significantly reduce polypeptide expression and/or activity.

The term “polynucleotide”, when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

“Antisense oligodeoxynucleotides” or “antisense oligonucleotides” (which terms are used interchangeably) are defined as nucleic acid molecules that can inhibit the transcription and/or translation of target genes in a sequence-specific manner. The term “antisense” refers to the fact that the nucleic acid is complementary to the coding (“sense”) genetic sequence of the target gene. Antisense oligonucleotides hybridize in an anti-parallel orientation to nascent mRNA through Watson-Crick base-pairing. By binding the target mRNA template, antisense oligonucleotides block the successful translation of the encoded protein. The term specifically includes antisense agents called “ribozymes” that have been designed to induce catalytic cleavage of a target RNA by addition of a sequence that has natural self-splicing activity (Warzocha and Wotowiec, “Antisense strategy: biological utility and prospects in the treatment of hematological malignancies.” Leuk. Lymphoma 24:267-281 [1997]).

The terms “vector”, “polynucleotide vector”, “construct” and “polynucleotide construct” are used interchangeably herein. A polynucleotide vector of this invention may be in any of several forms, including, but not limited to, RNA, DNA, RNA encapsulated in a retroviral coat, DNA encapsulated in an adenovirus coat, DNA packaged in another viral or viral-like form (such as herpes simplex, and adeno-associated virus (AAV)), DNA encapsulated in liposomes, DNA complexed with polylysine, complexed with synthetic polycationic molecules, conjugated with transferrin, complexed with compounds such as polyethylene glycol (PEG) to immunologically “mask” the molecule and/or increase half-life, or conjugated to a non-viral protein. Preferably, the polynucleotide is DNA. As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

The term “polypeptide”, in singular or plural, is used herein to refer to any peptide or protein comprising two or more amino acids joined to each other in a linear chain by peptide bonds. As used herein, the term refers to both short chains, which also commonly are referred to in the art as peptides, oligopeptides and oligomers, and to longer chains, commonly referred to in the art as proteins. Polypeptides, as defined herein, may contain amino acids other than the 20 naturally occurring amino acids, and may include modified amino acids. The modification can be anywhere within the polypeptide molecule, such as, for example, at the terminal amino acids, and may be due to natural processes, such as processing and other post-translational modifications, or may result from chemical and/or enzymatic modification techniques which are well known to the art. The known modifications include, without limitation, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. Such modifications are well known to those of skill and have been described in great detail in the scientific literature, such as, for instance, Creighton, T. E., Proteins—Structure And Molecular Properties, 2nd Ed., W. H. Freeman and Company, New York (1993); Wold, F., “Posttranslational Protein Modifications: Perspectives and Prospects,” in Posttranslational Covalent Modification of Proteins, Johnson, B. C., ed., Academic Press, New York (1983), pp. 1-12; Seifter et al., “Analysis for protein modifications and nonprotein cofactors,” Meth. Enzymol. 182:626-646 (1990), and Rattan et al., Ann. N.Y Acad. Sci. 663:48-62 (1992).

Modifications can occur anywhere in a polypeptide, including the peptide backbone, the amino acid side-chains and the amino or carboxyl termini. In fact, blockage of the amino or carboxyl group in a polypeptide, or both, by a covalent modification, is common in naturally occurring and synthetic polypeptides and such modifications may be present in polypeptides of the present invention, as well. For instance, the amino terminal residue of polypeptides made in E. coli, prior to proteolytic processing, almost invariably will be N-formylmethionine.

The term “recombinant” when used with reference to a cell, animal, or virus indicates that the cell, animal, or virus encodes a foreign DNA or RNA. For example, recombinant cells optionally express nucleic acids (e.g., RNA) not found within the native (non-recombinant) form of the cell.

As used herein, the term “β-amyloid precursor protein” β-APP) refers to a single membrane-spanning protein expressed in a wide variety of cells in many mammalian tissues, including the human polypeptide that is encoded by a β-APP gene localized in humans on the long arm of chromosome 21. Examples of specific isotypes of APP which are currently known to exist in humans are the 695-amino acid polypeptide described by Kang et al., Nature 325:733—736 (1987). A 751-amino acid polypeptide has been described by Ponte et al., Nature 331:525-527 (1988) and Tanzi et al., Nature 331:528-530 (1988). A 770-amino acid isotype of APP is described in Kitaguchi et al., Nature 331:530-532 (1988). A number of specific variants of APP have also been described having point mutations that can differ in both position and phenotype. A general review of such mutations is provided in Hardy, Nature Genet. 1:233-234 (1992). A mutation of particular interest is designated the “Swedish” mutation where the normal Lys-Met residues at positions 595 and 596 of the 695 form are replaced by Asn-Leu. This mutation is located directly upstream of the normal β-secretase cleavage site of APP, which occurs between residues 596 and 597 of the 695 form.

The terms “treat” or “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Chronic” administration refers to administration of the agent(s) in a continuous mode as opposed to an acute mode, so as to maintain the desired effect for an extended period of time.

“Intermittent” administration is treatment that is not consecutively done without interruption, but rather is cyclic in nature.

Administration “in combination with” one or more further therapeutic agents includes simultaneous (concurrent) and consecutive administration in any order.

An “individual” is a vertebrate, preferably a mammal, more preferably a human.

“Mammal” for purposes of treatment refers to any animal classified as a mammal, including humans, domestic and farm animals, and zoo, sports, or pet animals, such as dogs, horses, cats, cows, etc. Preferably, the mammal herein is human.

An “effective amount” is an amount sufficient to effect beneficial or desired therapeutic (including preventative) results. An effective amount can be administered in one or more administrations.

The terms “tumor” and “cancer” refer to any clinical condition characterized by unchecked and abnormal cell proliferation. The term “tumor” generally refers to any abnormal cell proliferation or tissue growth, including so called “benign” tumors that are unable to spread from their original focus, and “malignant” tumors that are capable of spreading beyond their anatomical site. The term “cancer” is generally used as a synonym for a malignant tumor.

The terms “selective expression” and “selectively expressed” are used to indicate that the expression level of the gene in the favored cell (e.g. tumor or cancer cell) is at least about 10-times, more preferably at least about 20-times, most preferably at least about 100-times higher than in other cells. The term “differential expression” is used to mean that there is at least about 2-fold, more preferably at least about 5-fold difference in the expression level of a gene in a cell of interest relative to a reference cell. The differential expression is called “over-expression” when the expression level of a gene is at least about 2-times, more preferably at least about 5-times higher is a “favored” cell than in other (reference) cells.

5.2 Modes of Carrying Out the Invention

5.2.2. Vector Construction

The invention involves the construction of polynucleotide comprising the coding sequence for a cleavage or enzyme recognition site, flanked by two caspase subunits. This polynucleotide is generally present as part of an expression vector, and is referred to as a “caspase cassette.” In a preferred embodiment, the caspase cassette encodes a fusion protein in which the original protease site between the two subunits of a native caspase is replaced by a different cleavage/enzyme recognition site.

The construction of the polynucleotides of the present invention conveniently starts with obtaining DNA encoding a native caspase from cDNA libraries prepared from tissue believed to possess the corresponding mRNA and to express it at a detectable level. For example, cDNA library can be constructed by obtaining polyadenylated mRNA from a cell line known to express the desired polypeptide, and using the mRNA as a template to synthesize double-stranded cDNA. The polynucleotide encoding a native caspase can also be obtained from a genomic library, such as a human genomic cosmid library.

Certain caspases are commercially available or may be obtained from various research laboratories. Alternatively, or in order to obtain caspases that are not commercially available, libraries, either cDNA or genomic, are screened with probes designed to identify the caspase gene of interest or the protein encoded by it. For cDNA expression libraries, suitable probes include monoclonal and polyclonal antibodies that recognize and specifically bind to the desired caspase. Anti-caspase antibodies are known in the art and some are commercially available. For example, a rabbit-antihuman caspase-9 monoclonal antibody is available from Serotec (Oxford, U.K., Catalog # AHP492), and a polyclonal rabbit anti-caspase-8 antibody is commercially available from Zymed (South San Francisco, Calif., U.S., catalog #51-1100). For cDNA libraries, suitable probes include oligonucleotide probes (generally about 20-80 bases) that encode known portions of a desired caspase, or suspected portions of a caspase not yet identified, and/or complementary or homologous cDNAs or fragments thereof that encode the same or a similar gene. Appropriate probes for screening genomic libraries include, without limitation, oligonucleotides, cDNAs, or fragments thereof that encode the same or a similar gene, and/or homologous genomic DNAs or fragments thereof. Screening the cDNA and genomic libraries with the selected probe may be conducted using standard protocols as described, for example, in Chapters 10-12 of Sambrook et al., Molecular Cloning: A Laboratory Manual. New York, Cold Spring Harbor Laboratory Press (1989).

Once DNA encoding a native caspase is synthesized, it can be altered by introducing amino acid substitutions, deletions and/or insertions. The sequence of the amino acid sequence variants of the caspase subunits used in the constructs of the present invention is at least about 80%, preferably at least about 90%, most preferably at least about 95% identical with the sequence of a corresponding native caspase. Such amino acid sequence variants can be produced by expressing the underlying DNA sequence in a suitable recombinant host cell, as described above, or by in vitro synthesis of the desired polypeptide. The nucleic acid sequence encoding a polypeptide variant of the present invention is preferably prepared by site-directed mutagenesis of the nucleic acid sequence encoding the corresponding native (e.g. human) polypeptide. Particularly preferred is site-directed mutagenesis using polymerase chain reaction (PCR) amplification (see, for example, U.S. Pat. No. 4,683,195 issued Jul. 28, 1987; and Current Protocols In Molecular Biology, Chapter 15, Ausbel et al., ed., 1991). Other site-directed mutagenesis techniques are also well known in the art and are described, for example, in the following publications: Current Protocols In Molecular Biology, supra, Chapter 8; Molecular Cloning: A Laboratory Manual., 2^{nd} edition (Sambrook et al., 1989); Zoller et al., Methods Enzymol. 100:468-500 (1983); Zoller & Smith, DNA 3:479-488 (1984); Zoller et al., Nucl. Acids Res., 10:6487 (1987); Brake et al., Proc. Natl. Acad. Sci. USA 81:4642-4646 (1984); Botstein et al., Science 229:1193 (1985); Kunkel et al., Methods Enzymol. 154:367-82 (1987), Adelman et al., DNA 2:183 (1983); and Carter et al., Nucl. Acids Res., 13:4331 (1986). Cassette mutagenesis (Wells et al., Gene, 34:315 [1985]), and restriction selection mutagenesis (Wells et al., Philos. Trans. R. Soc. London SerA, 317:415 [1986]) may also be used.

Amino acid sequence variants with more than one amino acid substitution may be generated in one of several ways. If the amino acids are located close together in the polypeptide chain, they may be mutated simultaneously, using one oligonucleotide that codes for all of the desired amino acid substitutions. If, however, the amino acids are located some distance from one another (e.g. separated by more than ten amino acids), it is more difficult to generate a single oligonucleotide that encodes all of the desired changes. Instead, one of two alternative methods may be employed. In the first method, a separate oligonucleotide is generated for each amino acid to be substituted. The oligonucleotides are then annealed to the single-stranded template DNA simultaneously, and the second strand of DNA that is synthesized from the template will encode all of the desired amino acid substitutions. The alternative method involves two or more rounds of mutagenesis to produce the desired mutant.

The replacement of an original protease site from a polynucleotide encoding a native caspase, or an amino acid sequence variant of a native caspase, is conveniently done using a method of overlapping PCR (Higuchi et al., Nucleic acids Res. 16:7351-7367 [1988]), especially if the X recognition site is not too long to be incorporated in the primers. However, a conventional method utilizing the presence of restriction sites can also be used for replacement of the recognition site. For PCR-based splicing, the desired cleavage/enzyme recognition sequence can be incorporated into the 3′ prime end of one caspase subunit and the 5′ prime end of the other caspase subunit.

The nucleic acid encoding the fusion polypeptides of the present invention can also be obtained by chemical synthesis, following known methods, such as the phosphoramidite method (Beaucage and Caruthers, Tetrahedron Letters 22:1859 [1981]; Matteucci and Caruthers, Tetrahedron Letters 21:719 [1980]; and Matteucci and Caruthers, J. Amer. Chem. Soc. 103: 3185 [1981]), and the phosphotriester approach (Ito et al., Nucleic Acids Res. 10: 1755-1769 [1982]).

In a particular embodiment, the caspase cassette is included in an expression vector, under control of an inducible promoter. A suitable inducible promoter is the MTII (metallothioneine-II) promoter (inducer: phorbol ester (TPA) and heavy metals), the MMTV (mouse mammary tumor virus) promoter (induced by glucocorticoids), ecdysone responsive promoter (induced by ecdysone and its derivatives) and the thyroid stimulating hormone a gene (induced by thyroid hormone) promoter. For further inducible promoters, and further details see: Gene Transfer and Expression: A Laboratory Manual, M. Kriegler editor, W. H. Freeman and Company, New York, 1990. There are a number of commercially available systems for inducible gene expression such as Tet-Off and Tet-On Gene Expression system from Clontech (Palo Alto, Calif.) based on tetracycline-inducible promoter.

The expression vectors of the present invention preferably are plasmids, but, especially for gene therapy, other vectors such as, retroviral vectors, adenovirus vectors, herpes viral vectors, and non-replicative avipox viruses are also contemplated, as disclosed, for example, in U.S. Pat. No. 5,174,993.

The vectors can be used to transform (mammalian) recombinant host cells, and the host cells can be cultured by methods well known in the art, such as those described in Sambrook et al., supra. Mammalian cells susceptible to caspase-induced apoptosis include, for example, monkey kidney CV1 cell line transformed by SV40 (COS-7, ATCC CRL 1651); human embryonic kidney cell line 293 (Graham et al, J. Gen. Virol. 36:59 [1977]); baby hamster kidney cells (BHK, ATCC CCL 10); Chinese hamster ovary (CHO) cells (Urlaub and Chasin, Proc. Natl. Acad. Sci. USA 77:4216 [1980]; monkey kidney cells (CV1-76, ATCC CCL 70); African green monkey cells (VERO-76, ATCC CRL-1587); human cervical carcinoma cells (Hela, ATCC CCL 2); canine kidney cells (MDCK, ATCC CCL 34); human lung cells (W138, ATCC CCL 75); human liver cells (Hep G2, HB 8065), and human breast carcinoma cells (MCF-7, ATCC HTB-22), the use of which is specifically exemplified.

5.2.3. Screening Assays

The screening assays of the present invention are preferably amenable to high-throughput screening of combinatorial and other chemical libraries, and are suitable for identifying polypeptide or small molecule drug candidates. Small molecules, which are usually less than 10K molecular weight, are desirable as therapeutics since they are more likely to be permeable to cells, are less susceptible to degradation by various cellular mechanisms, and are not as apt to elicit immune response as proteins. Small molecules include but are not limited to synthetic organic or inorganic compounds, and peptides. Many pharmaceutical companies have extensive libraries of such molecules, which can be conveniently screened by using the assays of the present invention. The assays can be performed in a variety of formats, including protein-protein binding assays, biochemical screening assays, immunoassays, cell based assays, etc. Such assay formats are well known in the art.

5.2.4. Gene Therapy

As noted before, the polynucleotides of the present invention can also be used in gene therapy. In gene therapy applications, genes are introduced into cells in order to achieve in vivo synthesis of a therapeutically effective genetic product, for example for replacement of a defective gene. Gene therapy includes both conventional gene therapy where a lasting effect is achieved by a single treatment, and the administration of gene therapeutic agents, which involves the one time or repeated administration of a therapeutically effective DNA or RNA.

There are a variety of techniques available for introducing nucleic acid into viable cells. The techniques differ depending upon whether the nucleic acid is transferred into cultured cells in vitro, or in vivo in the cells of the intended host. Techniques suitable for the transfer of the nucleic acid into mammalian cells in vitro include the use of liposomes, electroporation, microinjection, cell fusion, DEAE-dextran, the calcium phosphate method, etc. The currently preferred in vivo gene transfer methods include transfection with viral (typically retroviral) vectors and viral coat protein-liposome mediated transfection (Dzau et al., Trends in Biotechnology 11, 205-210 [1993]). In some situations it is desirable to provide the nucleic acid source with an agent that targets the specific cells, such as an antibody specific for a cell surface membrane protein on the target cells, a ligand for a receptor on the target cells, etc. Where liposomes are employed, proteins which bind to a cell surface membrane protein associated with endocytosis may be used to target and/or to facilitate uptake, e.g. capsid proteins or fragments thereof tropic for a particular cell type, proteins that target intracellular localization and enhance intracellular half-life. For review of gene marking and gene therapy protocols see Anderson et al, Science 256, 808-813 (1992).

5.3 Strategy for Isolating cDNA Clone(s) Encoding β-Secretase

Cells, which are susceptible to killing by the active caspase-3 and express endogenous β-secretase (practically all known eukaryotic cells characterized so far), such as MCF-7 cells, are transfected with a construct expressing suitable (e.g. Tet) regulator and are selected for stable transfectants using an appropriate marker. A clonal derivative of this (Y1) is then transfected with a construct that expresses a chimeric caspase cassette in which the original cleavage site between the large and small subunits is replaced by the recognition/cleavage site of β-secretase. Conditionally lethal clonal derivatives (Y2) are generated by using an inducible promoter to drive the expression of the chimeric caspase cassette. In this case, the expression of the cassette is under tight control of tetracycline repressor. The expression of caspase cassette is repressed in the continuous presence of tetracycline, and is induced by withdrawal of tetracycline. The proteolytic processing of the caspase precursor by endogenous β-secretase activates caspase and induces apoptotic cell death. Thus these cells will grow normally in the presence of tetracycline (uninduced condition) but would succumb to apoptosis in the absence of tetracycline (induced condition).

The clonal derivative of MCF-7 cells (Y2), as described above, is used for screening of cDNA expression library to isolate clones that potentially code for β-secretase. Y2 cells are transfected with cDNA expression library or are preferably transduced with retroviral cDNA expression library. When induced by withdrawal of tetracycline, majority of cells is expected to die as a result of processing and activation of caspase and ensuing apoptosis. However, a small number of clones may survive the induction conditions. The use of retroviral cDNA expression library provides a simple and convenient way of substantially enriching the clones at this stage by harvesting supernatant from individual clones that contain recombinant retroviral particles of interest and infecting fresh Y2 cells with them. These freshly infected cells are also induced as described earlier which will eliminate un-transfected cells as well as cells transfected with irrelevant cDNA clones. It is possible to use several rounds of enrichment. The total genomic DNA is prepared from these clones, and the cDNA inserts from integrated proviruses are amplified by PCR using primers derived from the flanking sequences of retroviral vectors. The amplified inserts are cloned and sequenced. The genetic suppressor elements thus identified are likely to fall in several categories:

1. Clones that contain cDNA insert coding for sense or antisense transcripts of the putative β-secretase gene. The expression of antisense transcript protects cells against apoptosis by significantly reducing or eliminating the level of β-secretase mRNA and/or protein, and thus preventing proteolytic processing/activation of caspase. Close examination of the insert sequence may reveal a putative open reading frame (ORF) (on the sense strand) and may also include certain evolutionarily conserved features of proteases in the putative ORF. It may also show signal sequence at the N-terminal end and trans-membrane domain(s). The cDNA insert sequence can be searched against nucleotide databases (such as GenBank, European Bioinformatics Institute [EBI] and DNA Database of Japan [DDJ]) in order to identify homology, if any, with existing sequences. If a potential ORF is found in the cDNA insert, it can be similarly searched against protein databases (such as GenBank, SwissProt, Protein Identification Resource [PIR] etc). These short inserts are then used as a probe to obtain full-length cDNA clones by conventional cloning methods based on hybridization.

Some clones imparting protection against caspase cassette induced apoptosis may code for sense transcripts of β-secretase. These clones will inevitably harbor a truncated cDNA insert often coding for a short fragment of polypeptide with potential to act as trans-dominant inhibitor. It is well recognized that proteins are made up of distinct functional modules some of which are capable of functioning even when they are taken out of context of the full-length polypeptide. Some of these domains, particularly those that are involved in multimerization (for instance, homo- or hetero-dimerization), have the potential to block the activity of the full-length protein, when co-expressed. Similarly, truncated versions of transcription factors coding only for DNA-binding domain have the potential to block the activity of the full-length transcription factor, when co-expressed. These are designated as trans-dominant inhibitors. As described above, these partial clones can be used to obtain full-length cDNA clone by conventional methods.

2. Some clones may not directly correspond to β-secretase. Nevertheless, they may have a potential to interfere with β-secretase activity. For example, they might code for proteins that negatively regulate the activity of β-secretase.

3. There may be yet another category of clones which eliminate the expression of β-secretase rather than its activity. This could be due to a block at any of the steps in the expression of gene such as transcription, post-transcription, translation etc.

4. The strategy will also lead to selection of the clones expressing antisense transcripts of caspase. These clones are obviously of no interest to the objective of the strategy, and are therefore not pursued further.

### 6. EXAMPLES

The invention is further illustrated by the following, non-limiting examples. Unless otherwise noted, all the standard molecular biology procedures are performed according to protocols described in (Molecular Cloning: A Laboratory Manual, vols. 1-3, edited by Sambrook, J., Fritsch, E. F., and Maniatis, T., Cold Spring Harbor Laboratory Press, 1989; Current Protocols in Molecular Biology, vols. 1-2, edited by Ausbubel, F., Brent, R., Kingston, R., Moore, D., Seidman, J. G., Smith, J., and Struhl, K., Wiley Interscience, 1987).

The Examples serve as an illustration of the invention through the description of the generation of a stable cell line with regulatable expression of a chimeric caspase cassette (Example 1); description of a strategy for the cloning of genes encoding β-secretase or other proteases following the present invention (Example 2); disclosure of an approach for cloning genes whose expression blocks the activity of a given enzyme (Example 3); and generation and characterization of a modified caspase-3, containing the cleavage site of β-secretase which plays a pivotal role in Alzheimer's disease (Example 4). Caspase-3 was chosen because it is a downstream caspase in the apoptotic pathway (Fernandes-Alnemri et al., J. Bio. Chem. 269:30761-30764 [1994]; Tewari et al., Cell 81:801-809 [1995]; Nicholson et al., Nature 376:37-43 [1995]). Overexpression of wild-type caspase-3 alone is not sufficient to cause cell death. However, the activated caspase-3, from either processing of its pro-form by upstream caspases or co-transfection of the individual subunits, is able to cause apoptosis of many cells. As discussed before, β-secretase is an important enzyme involved in the generation of amyloid β peptide from its precursor protein, APP. Processing of APP by β-secretase and other secretases has been extensively studied and several candidate β-secretases have been identified (Vassar et al., Science 286:735-741 [1999]; Yan et al., Nature 402:533-537 [1999]; Sinha et al., Nature 402:537-540 [1999]; Hussain et al., Mol. Cel. Neurosci. 14:419-427 [1999]; Lin et al., Proc. Natl. Acad. Sci. USA 97:1456-1460 [2000]). Example 4 shows that a modified caspase-3 containing β-secretase cleavage site induces cell death in a β-secretase dependent fashion.

### Example 1

Generation of a Stable Cell Line (Y2) with Regulatable Expression of a Chimeric Caspase Cassette

a. Genetic Engineering of a Construct for Constitutive and Inducible Expression of a Chimeric Caspase Cassette with a Known Enzyme Cleavage Sequence

An eukaryotic construct that expresses a chimeric caspase whose proteolytic processing is dependent on the availability of an enzyme X is generated. This is achieved by replacing the native protease site, present between the large and small subunit of caspase, with a recognition site for enzyme X. The resultant caspase is no longer subjected to the processing and activation by the upstream caspase involved in the normal cascade of proteolysis during induction of apoptosis. Instead its activation is now dependent on the recognition and cleavage by the enzyme X.

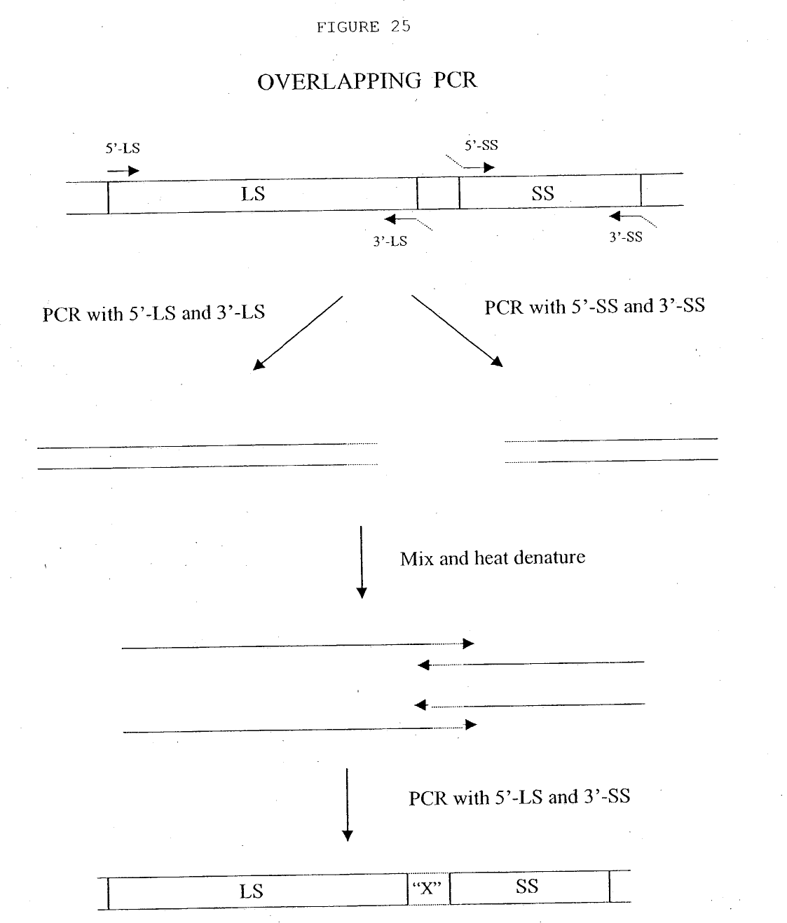

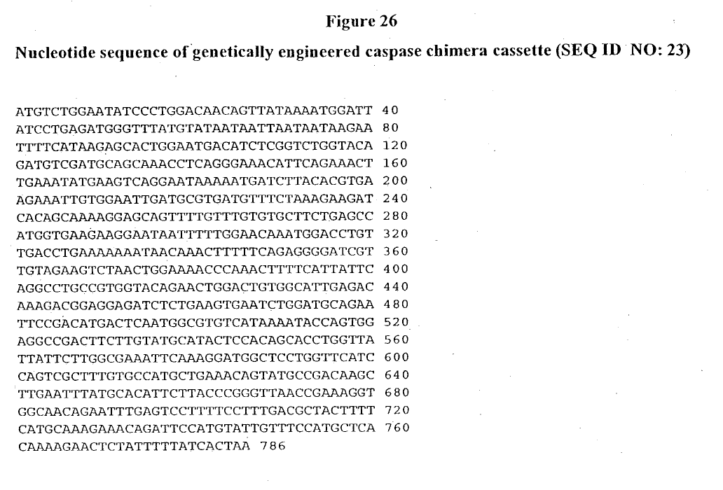

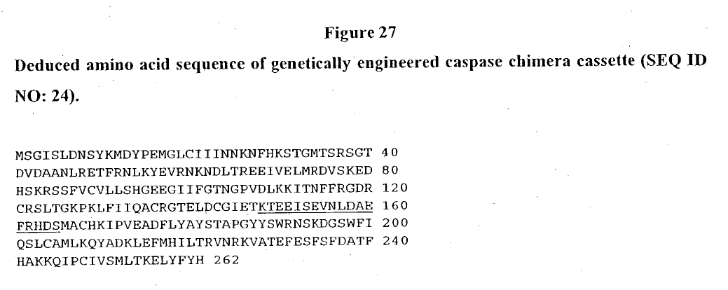

The replacement of a protease site as referred above is conveniently done using a method of overlapping PCR (Higuchi et al., Nucleic Acids Res. 16: 7351-7367 [1988]) especially if the X recognition site is not too long to be incorporated in the primers. However, a conventional method utilizing the presence of restriction sites can also be used for replacement of the recognition site. The template for PCR is a cDNA clone of caspase-3 (Tewari et al., Cell 81: 801-809 [1995]) obtained from the Univ. of Michigan. Although this procedure describes the use of caspase-3, any suitable caspase, especially the execution caspases that are involved in late phase of caspase cascade, which do not exhibit appreciable extent of auto-processing and auto-activation, can be substituted. The organization of large subunit (LS) and small subunit (SS) with respect to the upstream caspase recognition site in caspase polypeptide is N-LS-protease site-SS-C, wherein N and C refer to the amino- and carboxyl-terminus of polypeptide respectively. (It is noted, however, that a cassette, in which the position of the large and small subunits is reversed [N-SS-protease site-LS-C] may be also suitable.) The overlapping PCR (see schematic diagram in FIG. 25) uses a set of four primers: 5′-LS and 3′-LS are sense and antisense primers complementary to the 5′- and 3′-ends of the LS reading frame respectively; 5′-SS and 3′-SS are sense and antisense primers complementary to the 5′- and 3′-ends of the SS reading frame respectively. The 5′-LS and 3′-SS primers incorporate a recognition site for an appropriate restriction enzyme in order to facilitate the cloning of the final PCR product into a suitable eukaryotic expression vector. The 3′-LS primer contains a sequence coding for the recognition site for enzyme X at the 5′-end of the primer and therefore it would not hybridize to the template. Similarly, the 5′-SS primer contains the sequence coding for the X recognition site at the 5′-end of the primer.

Two separate sets of PCR reactions are performed using the same template (for example caspase-3), one with 5′-LS and 3′-LS primers and another with 5′-SS and 3′-SS primers. These initial PCR reactions amplify the coding sequences for large and small subunits of caspase separately. The products of these PCR reactions are then purified, mixed, heat denatured and subjected to the second PCR reaction in the presence of 5′-LS and 3′-SS primers. An internal priming takes place in this PCR reaction due to complementarity at the 3′-end of the LS PCR product and 5′-end of the SS PCR product as a result of the incorporation of X recognition sequence in the 3′-LS and 5′-SS primers. The final PCR product is digested with the restriction enzyme for which the recognition site was incorporated into the 5′-LS and 3′-SS primers, and cloned into an eukaryotic expression vector. Any eukaryotic expression vector with appropriate promoter/enhancer elements can be used, for example pcDNA3.1 (Invitrogen, San Diego, Calif.) that uses immediate early promoter of human cytomegalovirus (HCMV).

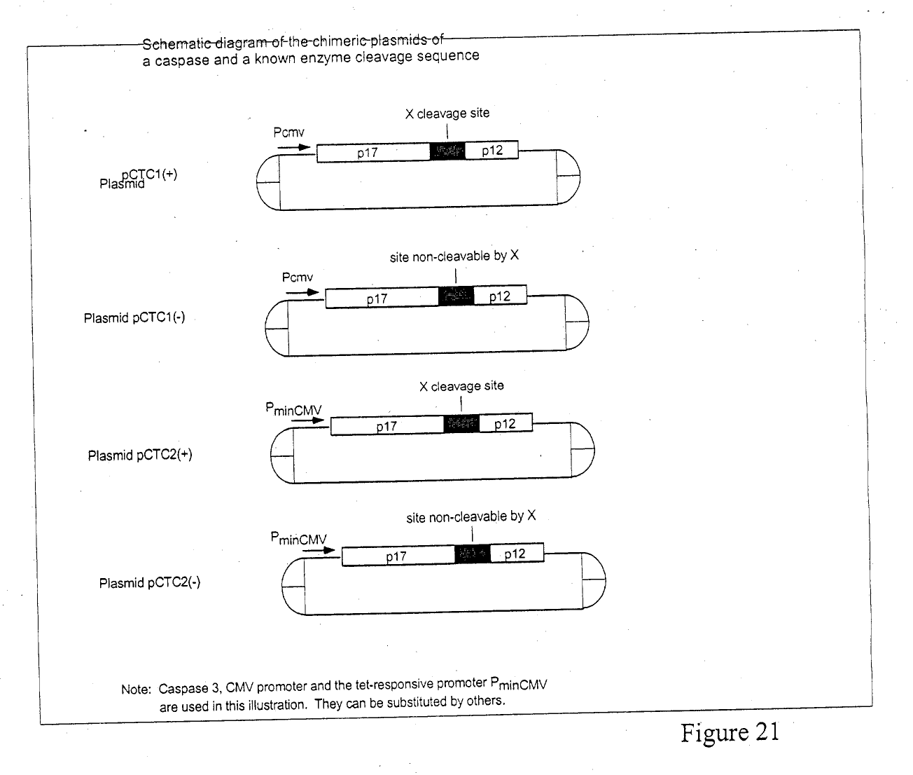

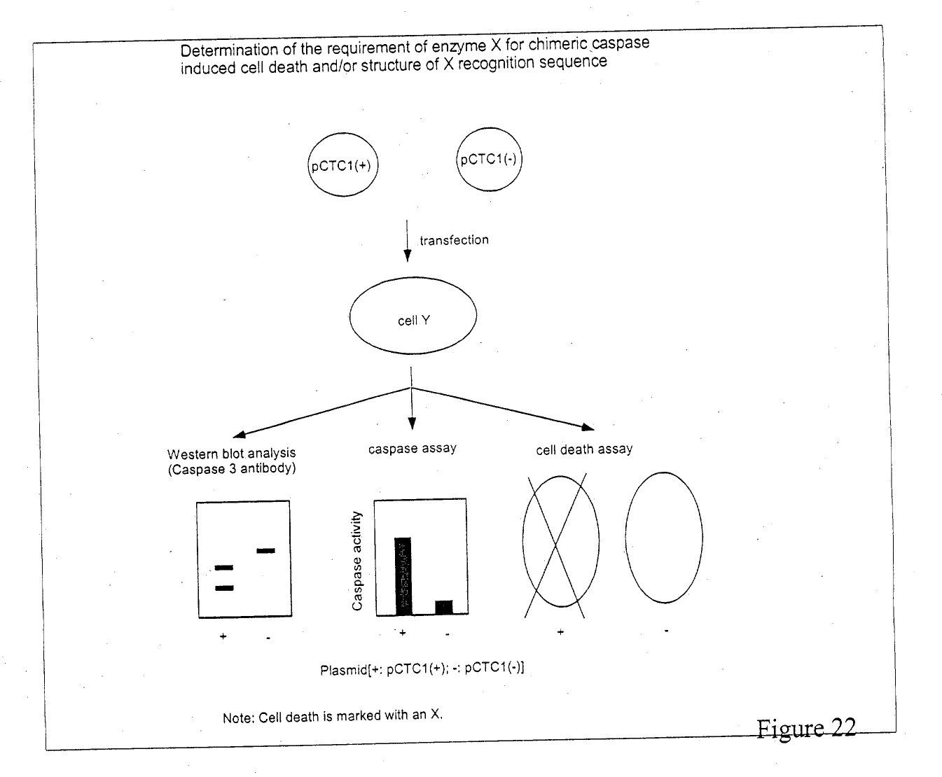

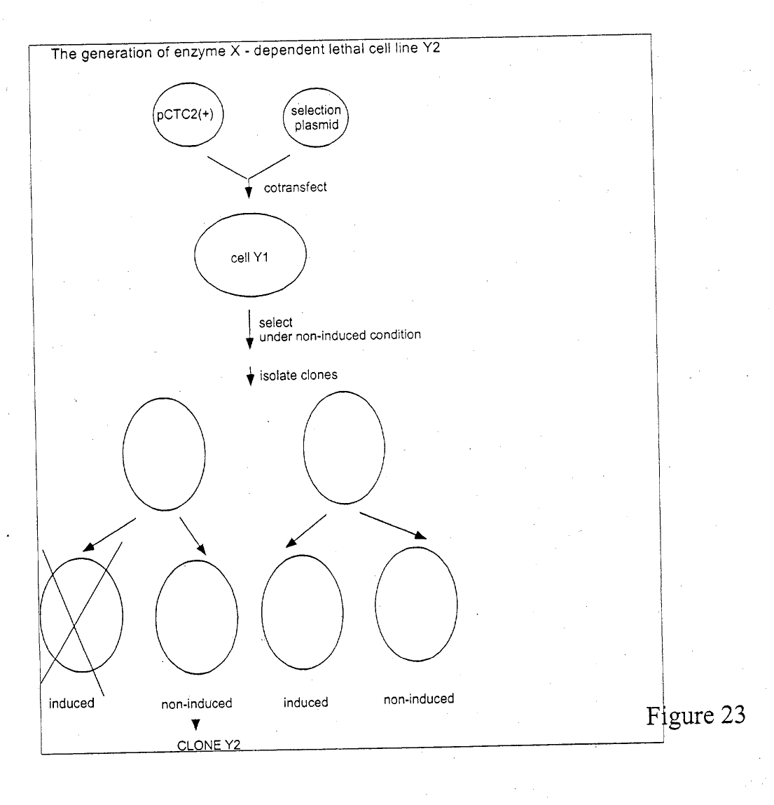

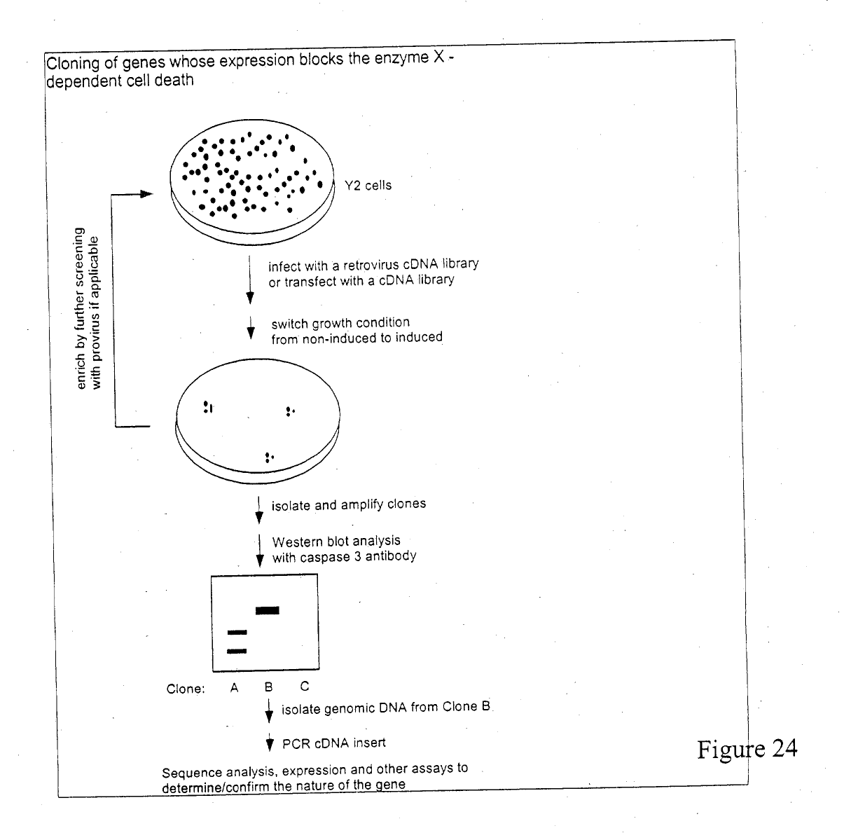

As shown in FIG. 21, the above mentioned strategy gives rise to a construct, e.g. pCTC1 (+), that expresses caspase chimera constitutively and wherein the processing/activation of the chimera is dependent on the enzyme X. As a control, a mutant site that is not recognized and cleaved by enzyme X is substituted for the wild type (cleavable) site, e.g. pCTC1 (−) in FIG. 21. Although the example refers to a vector with CMV promoter, any vector containing eukaryotic promoter/enhancer elements and RNA processing signals can substitute it. Since it is envisaged that the strategy to clone enzyme X or inhibitors of enzyme X will depend upon the generation of conditionally lethal cell line, another set of eukaryotic constructs with inducible promoter are made, e.g. pCTC2 (+). For this purpose, a vector with inducible promoter is used, such as pTRE (Clontech, Palo Alto, Calif.) which contains minimal promoter from HCMV modified by incorporation of tetracycline operator (tetO) sequences (also designated as tetracycline-responsive element) in order to make it tetracycline-inducible. Once again, a control construct is made in which wild type recognition site for enzyme X is replaced by a non-cleavable mutant site, e.g. pCTC2 (−), as shown in FIG. 21. The tetracycline-inducible system (Gossen and Bujard, Proc. Natl. Acad. Sci. USA 89: 5547-5551 [1992]), exemplified in FIG. 21, can be replaced by any other suitable inducible system. For example, steroid- or metallothionein-inducible transcriptional regulatory elements can also be used.

b. Determination of the Requirement of Enzyme X for Apoptotic Cell Death Induced by Chimeric Caspase

The ability of the chimeric caspase construct to induce apoptotic cell death when introduced into cells, depends on the availability of the enzyme X in that cell type and whether or not the cell is susceptible to the caspase activity. This is determined by transient transfection of cells with the constitutively expressing construct e.g. pCTC1 (+), and examining processing and activation of caspase and induction of apoptotic cell death as shown schematically in FIG. 22. As a control, cells are transfected with pCTC1 (−) construct that lacks a cleavable site for enzyme X. Cells are transiently transfected by any of the standard methods such as calcium phosphate, DEAE-dextran or lipofection. The use of specific inhibitors of enzyme X, if known and available, would help establish authenticity of the cleavage of caspase cassette by the enzyme X. A positive control for the strategy is the use of two separate constructs expressing large and small subunits of the chosen caspase. Co-transfection of cells with these constructs induces apoptosis if the transfected cells are susceptible to apoptosis by the caspase.

c. Processing of the Chimeric Caspase Cassette

The cleavage of the caspase cassette is examined by Western blot using a standard method. The extract prepared 24-48 h post-transfection from cells transfected with pCTC1 (+) or pCTC1 (−) is electrophoretically resolved under denaturing conditions on low percentage SDS-PAGE gel, proteins transferred onto nitrocellulose, nylon or PVDF membrane, and probed with antibodies specifically recognizing epitopes on caspase. If the cells express enzyme X, introduction of pCTC1 (+) should result in the processing of the caspase cassette as evident by the appearance of two faster migrating bands (17 kDa and 12 kDa in the case of caspase-3) corresponding to the two subunits of caspase. These fragments may be more easily detectable if the transfected cells are treated with caspase inhibitors such as z-VAD.fmk to block turnover of the proteins. The control cells, transfected with pCTC1 (−), should reveal only unprocessed caspase chimera (39-43 kDa). The precise molecular weight of the species, unprocessed and processed, along with recognition by the specific antibodies helps to ascertain authentic cleavage of the precursor. Additionally, cells are transfected with a construct that expresses native caspase (i.e. with its own cleavage site), induced to undergo apoptosis, and the cell extract subjected to Western blotting side by side. Such a sample provides markers for the unprocessed caspase and the small and large subunits of the processed caspase, as well as helps in establishing the authenticity of the cleavage by enzyme X. Another method to generate markers for the large and small subunits of the caspase is to transfect cells with two separate constructs expressing the large and small subunits and perform Western analysis on the cell extract derived from the transfected cells.

d. Measurement of the Caspase Activity

The protease activity of the caspase is also directly monitored in the cell extracts prepared as above. The ApoAlert® Caspase-3 assay kit, available commercially from Clontech, allows convenient fluorometric or colorimetric detection and assay of caspase-3. For example, the fluorescent kit detects the emission shift of 7-amino-4-trifluoromethyl coumarin (AFC). The AFC-substrate conjugate, DEVD-AFC, usually emits blue light (λmax=400 nm). However, upon proteolytic cleavage of the substrate by Caspase-3, free AFC fluoresces yellow-green (λmax=505 nm). Thus monitoring of emission at 505 nm in the cell extract, in relation to the uninduced control extract, allows determination of caspase activity. The activity can also be quantitated accurately and reproducibly using a standard curve established with free AFC. As described above, if there is endogenous expression of enzyme X in these cells, the caspase activity is generated in cells transfected only with pCTC1 (+) but not with pCTC1 (−).

e. Demonstration of Apoptosis

The cells are examined 18-48 h after transfection for induction of apoptotic cell death. The microscopic examination reveals tell-tale signs of apoptosis including cell swelling, fragmentation of nuclei, rupture of nuclear membrane, ghost nuclei, vacuolization of cytoplasm and cells undergoing lysis. When genomic DNA prepared from the cells undergoing apoptosis is electrophoresed on agarose gel, it shows a typical ladder-like appearance indicating digestion of chromosomal DNA at inter-nucleosomal sites by nucleases activated during apoptosis. All these features are absent in cells transfected with pCTC1 (−). The chromosomal DNA degradation induced during apoptotic cell death can also be documented in situ by TUNEL (terminal deoxynucleotidyl transferase-mediated dUTP nick end labeling) method (in situ death detection kit from Boehringer Mannheim). In this method, cells are permeabilized and incubated with terminal deoxynucleotide transferase (TdT), suitable buffer and modified nucleotides, X-dUTP where X is a tag such as biotin, fluorescein or digoxegenin. This enzyme is a template-independent DNA polymerase and it uses dNTPs to incorporate nucleotides at 3′-OH groups of DNA strands. Since 3′-ends are generated by nucleases activated during apoptosis, only apoptotic cells incorporate tagged dUTP. The incorporated tag can be revealed by appropriate method. For example, ApopTag kit (Oncor) uses digoxegenin-dUTP as a substrate for TdT. The digoxegenin labeled DNA is detected by the use of anti-digoxegenin antibodies conjugated to enzyme such as peroxidase.

f. The Generation of Enzyme X-Dependent Conditionally Lethal Cell Line Y2

Once a cell line (Y) is identified that expresses the enzyme of interest (X) and undergoes apoptosis in response to the activation of a chosen caspase, stable cell lines (Y2) conditionally expressing the caspase cassette are generated with the aid of an inducible expression system. A two step process accomplishes this:

g. Generation of a Stable Cell Line (Y1) Expressing the Regulator of an Inducible System

The procedure is described utilizing the tetracycline-inducible system only for illustrative purpose. Any other suitable inducible system can be used for the purpose. A cell line (Y) that expresses enzyme X and undergoes apoptosis in response to activation of the caspase is used as a starting cell line for serial derivation of stable cell lines Y1 and Y2. The cells are co-transfected with a plasmid expressing the tetracycline regulator and another plasmid expressing a selectable marker such as Neo^{r} (neomycin phosphotransferase) that provides resistance to a drug like neomycin (G418). The use of a commercially available construct, pTet-Off™ (from Clontech), obviates the need for two plasmids. pTet-Off™ expresses tetracycline-controlled transactivator (tTA), a fusion of 1-207 amino acid residues of tetracycline repressor (TetR) and the C-terminal activation domain (130 amino acid residues) of the VP16 transactivator of herpes simplex virus (HSV), as well as Neo^{R} selectable marker. Stably transfected cells are selected by exposing cells to increasing concentration of the drug. The careful titration should allow to essentially eliminate all the un-transfected cells which are susceptible to the drug. The individual clones are isolated using standard methods such as limiting dilution or using cloning cylinders. Several clones are isolated and analyzed in order to select clones that express highest levels of Tet repressor, which will ensure tight regulation i.e. minimal, or no expression of Tet-inducible gene in the presence of tetracycline. The final clones chosen are examined for their Tet-responsiveness by transfecting with a plasmid containing a reporter gene under the control of Tet-inducible promoter. The use of green fluorescent protein (GFP) as a reporter (e.g. pBI-EGFP construct driving the expression of enhanced GFP from tet-inducible promoter; available from Clontech) provides an easy and convenient way to examine the ability of cells to effectively repress Tet-inducible genes. The ideal clones exhibit green fluorescence only in the absence of tetracycline. The clones, which show no fluorescence at all (above the autofluorescence shown by some cell lines in the presence of tetracycline), are ideally suited for further study. The tight control will ensure that apoptosis is not induced in the presence of tetracycline in Y2 cells.

h. Generation of a Stable Cell Line (Y2) with Regulatable Expression of a Chimeric Caspase Cassette

The Y1 cells are co-transfected with a Tet-responsive plasmid containing the chimeric caspase cassette, e.g. pCTC2 (+), and another plasmid expressing a selectable marker different from the one used in the first selection. A number of selectable markers are available such as hygromycin B phosphotransferase, thymidine kinase, dihydrofolate reductase etc. The cells are selected with increasing concentration of an appropriate drug, clones expanded and tested for induced apoptosis. It is important to note that cells are always kept in a medium supplemented with appropriate concentration of tetracycline during the entire procedure of transfection, selection, maintenance and expansion. The cells are also kept under selection pressure using an appropriate concentration of the selection drug in order to prevent the loss of a transfected gene. For testing purpose, an aliquot of cells is taken, washed with PBS and resuspended in fresh medium lacking tetracycline. Cells are examined at various time points for signs of apoptotic cell death as described earlier. The clones that grow normally under uninduced condition (presence of tetracycline) but undergo apoptosis when induced (withdrawal of tetracycline) are selected for further studies. These clones (Y2) are said to exhibit conditional lethal phenotype that is essential for the strategy of cloning novel proteases or their inhibitors.

### Example 2

Cloning of Genes Encoding β-Secretase or Novel Proteases Using the Approach

Proteolytic cleavage plays a critical role in cellular processes and consequently is an important target in the therapeutic development. This is exemplified by β-secretase and γ-secretase, which are involved in the generation of amyloid β peptide (Aβ). Inhibition of the secretase processing is being pursued as an avenue for the treatment of Alzheimer's disease. Identification of these enzymes and isolation of corresponding genes coding for the enzymes would significantly increase the likelihood to reach this goal. Neither the gene coding for β-secretase has been cloned nor the protein has been purified to homogeneity for structural or enzymatic studies. Although many putative clones have been described, none of them have been yet shown to be authentic. However, the cognate recognition/cleavage sequence for β-secretase in β-amyloid precursor protein (βAPP) has been defined. The cloning strategy proposed herein requires minimal information about the target protease. The only information that is required for successful implementation of the strategy is the substrate recognition/cleavage sequence. As discussed earlier, the native cleavage site of a chosen caspase is substituted by the cognate recognition sequence of the target protease.

For illustration purpose, a strategy to isolate cDNA clone(s) encoding β-secretase is described. MCF-7 cells, which are susceptible to killing by the active caspase-3 and express endogenous β-secretase, are used to serially derive Y1 and Y2 clones as described in the previous section. A construct that expresses a chimeric caspase cassette in which the recognition/cleavage site of β-secretase replaces the original cleavage site between the large and small subunits is used for the purpose. A mutant β-secretase cleavage sequence ( . . . ⁻¹⁰KTEEISEVNL_DAEFRHDS⁺⁸ . . . ) (SEQ ID NO: 22), found in certain families with an autosomal dominant form of Alzheimer's disease, was chosen in order for the efficient cleavage of the chimeric caspase cassette. Two residues at the cleavage site are found mutated in this sequence, Lys to Asn at residue 595 and Met to Leu at residue 596. This mutation in β-amyloid precursor protein (β-APP) has been shown to increase the production of amyloid β-protein (Aβ) by ˜6-8 fold in vitro (Citron et al., Nature, 360, 672-674, [1992]). The cleavage sequence was concluded with S⁺⁸ to avoid selecting for the minor cleavage known to occur immediately after this residue (Higaki et al., Neuron 14, 651-659, [1996]). A control caspase cassette with a non-cleavable β-secretase site (Citron et al., Neuron 17, 171-179, [1996]) is also prepared. For comparison sake, a construct with the wild type cleavage site ( . . . ⁻¹⁰KTEEISEVKM_DAEFRHDS⁺⁸ . . . ) (SEQ ID NO: 21) is also made. Conditionally lethal clonal derivatives (Y2) are generated using a tetracycline-inducible promoter to drive the expression of the chimeric caspase cassette. The expression of caspase cassette is repressed in the continuous presence of tetracycline, and is induced by withdrawal of tetracycline. The proteolytic processing of the caspase precursor by endogenous β-secretase activates caspase and induces apoptotic cell death. Thus these cells grow normally in the presence of tetracycline (uninduced condition) but undergo apoptosis in the absence of tetracycline (induced condition).

The conditionally lethal clonal derivative of MCF-7 cells (Y2), as described above, is used for screening of cDNA expression library to isolate clones that potentially code for β-secretase. Y2 cells are transfected with cDNA expression library made in a conventional plasmid vector or retroviral vector. When induced by withdrawal of tetracycline, majority of cells is expected to die as a result of processing and activation of caspase and ensuing apoptosis. However, a small number of clones may survive the induction conditions. The use of retroviral cDNA expression library provides a simple and convenient way of substantially enriching the clones at this stage by harvesting supernatant from individual clones that contain recombinant retroviral particles of interest and infecting fresh Y2 cells with them. These freshly infected cells are also induced as described earlier which will eliminate un-transfected cells as well as cells transfected with irrelevant cDNA clones. It is possible to use several rounds of enrichment. The total genomic DNA is prepared from these clones and the cDNA inserts from integrated proviruses are amplified by PCR using primers derived from the flanking sequences of vectors. The amplified inserts are cloned in suitable vector and sequenced by dideoxy chain termination method. The strategy essentially leads to the isolation of genetic suppressor elements that rescue cells from apoptosis induced by the activation of caspase cassette. The elements thus identified fall in several categories. Some clones contain antisense transcripts derived from the putative β-secretase cDNA. The expression of antisense transcript protects cells against apoptosis by significantly reducing or eliminating the level of β-secretase mRNA and/or protein, and thus preventing proteolytic processing/activation of caspase. Close examination of the insert sequence may reveal a part of the ORF (on one of the strands) and may also include certain evolutionarily conserved features of proteases in the putative ORF. These short inserts are then used as probe to obtain full-length cDNA clones by conventional cloning methods based on hybridization. Some clones contain sense transcripts of β-secretase that code for partial domains of protein with potential to act as trans-dominant derivatives. As described above, these partial clones can be used to obtain full-length cDNA clone by conventional methods.

The putative clones of β-secretase can be validated by a number of approaches. For example, co-expression of the putative β-secretase clone with a construct that directs the expression of β-APP should lead to correct processing of the latter, which can be examined by immunoprecipitation or Western blotting of the transfected cell extract using monoclonal antibodies recognizing the epitope(s) on Aβ peptide. Any eukaryotic host can be used for this purpose, such as mammalian cell line (e.g. MCF-7), insect cell line (e.g. Sf-9) or yeast (e.g. Saccharomyces cerevisiae). The specificity of the substrate recognition and cleavage can be easily monitored by using β-APP with mutant cleavage sites. For example, β-APP with Swedish mutant cleavage site should exhibit enhanced processing. Similarly, a non-cleavable site can be used to test how closely the putative β-secretase clone mirrors the specificity of the authentic native enzyme. A complementary approach is to conduct a similar specificity assay in vitro using bacterially expressed and purified recombinant enzyme protein as well as a substrate polypeptide (β-APP). Specific antibodies, polyclonal or monoclonal, can be raised using bacterially expressed and purified β-secretase or synthetic peptide(s) derived from the ORF as immunogens. These antibodies can be used to validate the authenticity of the putative β-secretase clone(s) by testing whether they immunoprecipitate native β-secretase activity from cells. Similarly, these antibodies can be tested for their ability to immuno-deplete native β-secretase activity. An eukaryotic construct made to express antisense transcript of β-secretase, by cloning the candidate cDNA in such a way as to transcribe non-coding strand, can be examined for its ability to suppress endogenous β-secretase activity in transiently transfected cells. It is not necessary to use a full-length cDNA for this purpose. A partial cDNA encompassing the translation initiation site usually suffices for effective suppression of MRNA and/or protein levels. Another approach to unequivocally validate the β-secretase clone(s) is to generate knock out mouse by targeted disruption of the endogenous β-secretase locus, and testing for β-secretase activity in various tissues.

The baculovirus system offers a convenient method for high level expression of heterologous protein in eukaryotic cells (O'Reilley et al., Baculovirus expression vectors: A Laboratory Manual, Oxford University Press, Oxford, 1994). Once a full-length clone of β-secretase is isolated and validated, the cDNA insert is subcloned under the polyhedrin promoter of a baculovirus vector. The resultant construct is cotransfected into appropriate insect cells, such as Sf9 cells (derived from Spodoptera frugiperda insect, ATCC CRL 1711), along with helper virus. A recombination event takes place between the helper virus and the cDNA insert present in the construct, leading to the generation of recombinant baculovirus. The latter is isolated from plaques, amplified and used for scale up of protein expression. The polyhedrin promoter is activated during the late stage of virus replication and offers a high level of transcription of a linked gene. A particular advantage of insect cells is the proper post-translation modification and processing of heterologous eukaryotic proteins which is often required for biological activity of the recombinant protein. β-secretase, expressed and purified thus, can be used for structural or functional studies. A number of baculovirus systems are available commercially such as, for example, Bac-N-Blue™ (Invitrogen), BacPAK™ (Clontech), BAC-TO-BAC™ (Life Technologies), Bac Vector System (Novagen) etc.

### Example 3

Cloning of Genes Whose Expression Blocks the Activity of Enzyme X

The strategy described in the Example 2 also permits the isolation of clones that code for proteins with novel activity of inhibiting β-secretase. The inhibition of endogenous β-secretase by the products of these cDNA inserts prevents processing/activation of the caspase cassette and thereby rescues the clones from induction of cell death. Isolation and characterization of such potential inhibitors of β-secretase will have tremendous application in the treatment of Alzheimer's disease. Additionally, it will also reveal components involved in the regulation of such an important activity.

### Example 4

Construction and Characterization of a Modified Caspase-3 Containing β-Secretase Cleavage Site

Materials and Methods

Cells and reagents Human embryonic kidney 293T cells were maintained in Dulbecco's Modified Eagle Medium (DMEM) supplemented with 10% heat-inactivated fetal bovine serum. VAD.fmk, an ICE family protease inhibitor, was from Clontech (Palo Alto, Calif.).

Caspase-3 constructs All constructs were made using the pcDNA3 vector wherein the expression of caspase genes is under the control of CMV promoter. Plasmid pCSP3 contains the full-length caspase-3 coding region, and plasmid pCBC1 contains a modified caspase-3 where the large and small subunits of caspase-3 flank a 19 amino acid sequence corresponding to the β-secretase cleavage site of APP of the Swedish version. This modified caspase-3 gene was constructed by a two-step PCR approach (Higuchi et al., Nucleic Acids Res. 16: 7351-7367 [1988]) using human caspase-3 gene as a template and two sets of primers as following. The first set of primers, 5′-GCGGGATCCATGTCTGGAATATCCCTGGACAAC-3′ (SEQ ID NO: 25) and 5′-CTGCATCCAGATTCACCTCAGAGATCTCCTCCGTCTTTGTCTCAA TGC CACAGTCC-3′ (SEQ ID NO: 26), was used to amplify the large subunit of caspase-3. The second set of primers, 5′-TGAGGTGAATCTGGATGCAGAATTCCG ACATGACTCAATGGCGTGTCATAAAATAC-3′ (SEQ ID NO: 27) and 5′-CGCTCTAGATTAGTGATAAAAATAGAGTTCTTTTGTG-3′ (SEQ ID NO: 28), was designed to amplify the small subunit of caspase-3. In addition, the 3′ end primer of the first set and the 5′ end primer of the second set were designed to contain sequences complementary to each other.

Apoptosis assays 293T cells were seeded onto 60 mm dishes at a density of 1.5×10⁶ cells and, the next day, were transiently transfected with 2.5 μg plasmid DNA mixed with 15 μl lipofectamin reagent (Life Technologies, Grand Island, N.Y.). Twenty-four or 48 h after transfection, cell death was analyzed by one of the following methods. For the first assay, apoptosis was quantitated by visual inspection of cells. Cells with a shrunken, rounded, detached or blebbed appearance were scored as apoptotic. Usually 3 or more random fields were photographed and counted, and each field contained at least several hundred cells. In the case of co-transfection with green fluorescent protein (GFP)-expressing plasmid, cells were examined with an inverted fluorescent microscope, and only green cells with apoptotic features were counted. The second apoptotic assay used was the detection of nucleosomal ladder in apoptotic cells using ApoAlert LM-PCR ladder assay kit from Clontech. The procedure recommended by Clontech was followed.

Caspase-3 activity assay Caspase-3 activity was determined following the instructions of ApoAlert CPP32/Caspase-3 Fluorescent Assay kit (Clontech).

Site-directed mutagenesis Caspase-3 and BACE mutants were generated through site-directed mutagenesis using the protocol of QuickChange method (Stratagene, La Jolla, Calif.), and were confirmed by DNA sequencing.

Western blot analysis Cells were collected in PBS and immediately lysed by the addition of an equal volume of 2×SDS loading buffer. After boiling and centrifugation, supernatants containing equal amount of proteins were loaded onto 14% SDS-PAGE gels. The proteins were resolved and transferred to a nylon membrane, and probed with antibodies against human caspase-3 (Santa Cruz Biotech, Santa Cruz, Calif.).

β-NTF assay 293T cells were seeded onto 60 mm dishes at a density of 1.5×10⁶ cells and, the next day, were transiently transfected with 2.5 μg plasmid DNA mixed with 15 μl lipofectamin reagent (Life Technologies, Grand Island, N.Y.). In the case of transfection with pAPPsw plasmid, 0.5 μg pAPPsw was mixed with 2.0 μg carrier plasmid (unrelated plasmid). Twenty-four or 48 h after transfection, medium was collected and determined for P-NTF by ELISA method as described [Liu, 2000]

Results

A modified caspase-3 construct was made by replacing the interdomain linker with a β-secretase cleavage site, a 19 amino acid sequence derived from APP, ⁻¹¹TKTEEI SEVNLDAEFRHDS⁺⁸ (SEQ ID NO: 29), where β-secretase cleavage takes place between leucine and aspartate residues at P₋₁ and P₁ positions respectively. The rationale for this selection is as following. First, previous studies have shown that there does not appear to be cleavages within this region by proteases other than β-secretase (Higaki et al., Neuron 14: 651-659 [1995]). Second, a statin inhibitor that covers P₋₁₀ to P₄ has been shown to specifically inhibit β-secretase activity and to associate with BACE (β-site APP cleaving enzyme) (Sinha et al., Nature 402: 537-540 [1999]), suggesting that this region is likely to contain necessary information for the recognition by β-secretase. Finally, the Swedish version of APP was used because it is more easily processed by β-secretase (Citron et al., Nature 17: 672-674 [1992]; Cai et al., Science 22: 514-516 [1993]).

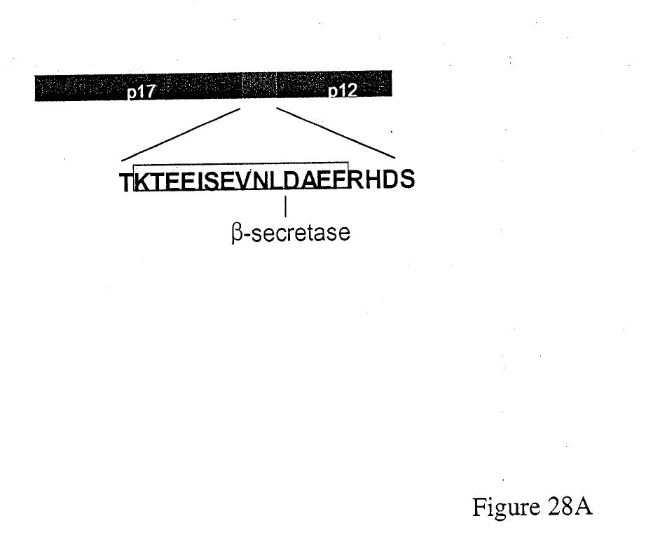

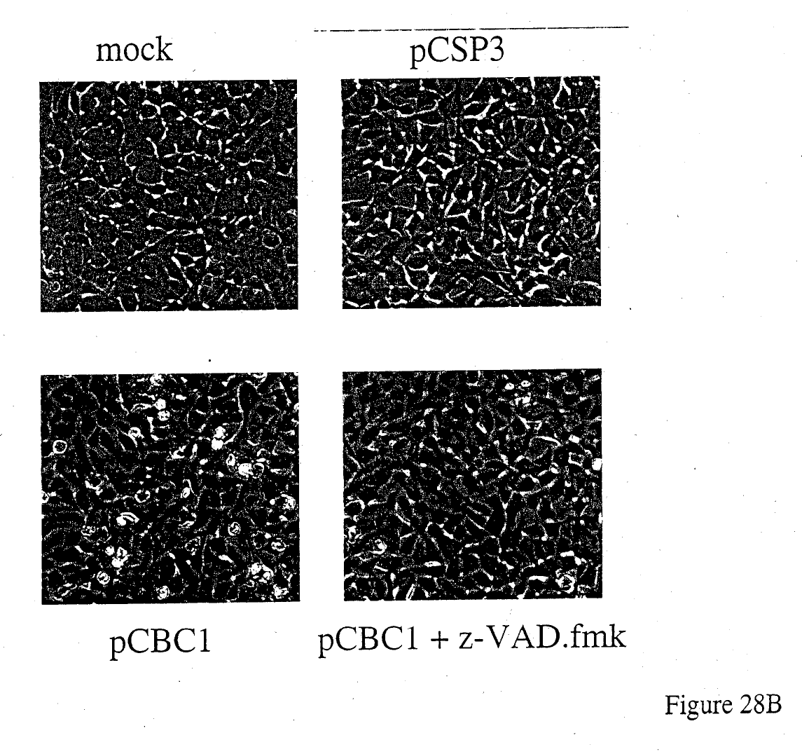

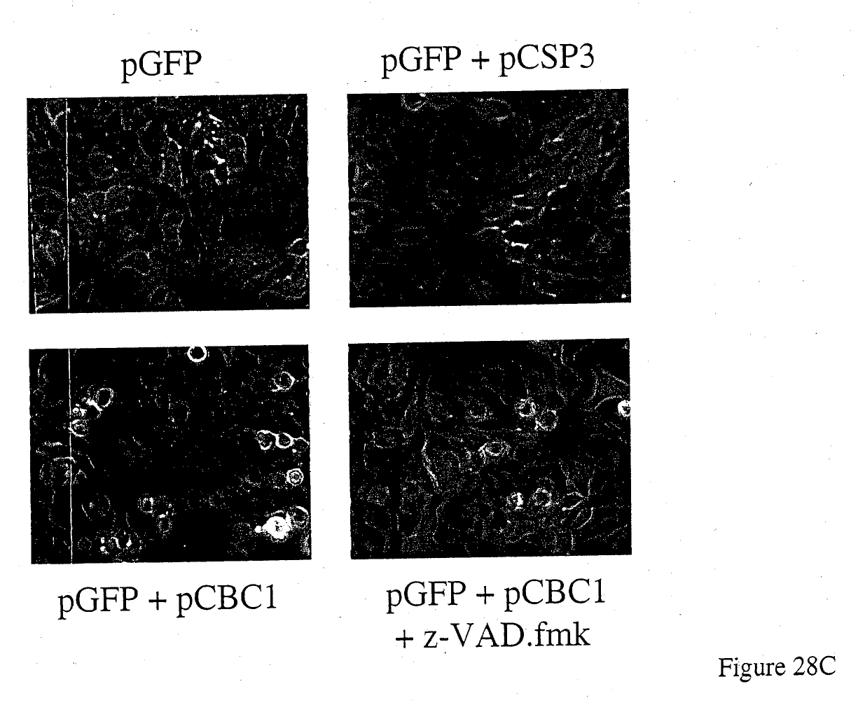

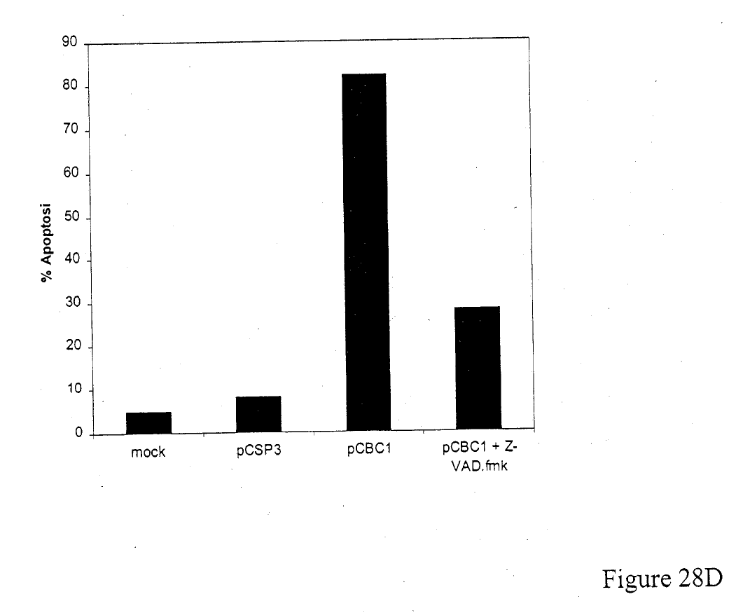

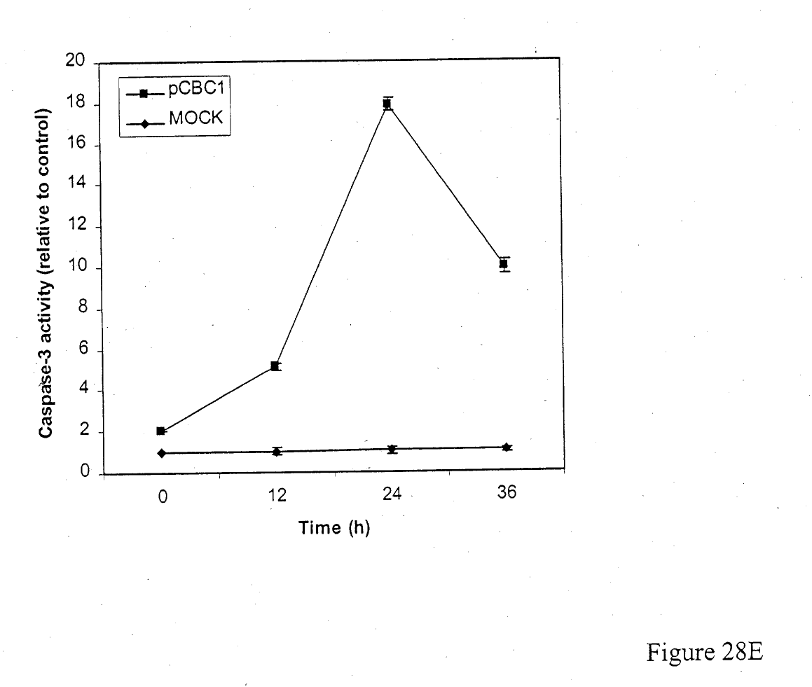

A two-step PCR process (Higuchi et al., supra [1988]) was used to construct a modified caspase-3 gene in such a way that the large and small subunits of caspase-3 were fused in-frame with the sequence encoding the 19 amino acids of APP (FIG. 28A). The modified caspase-3 gene was then subcloned into pcDNA3.1 expression vector to give rise to plasmid pCBC1. 293T cells were transfected with pCBC1 and effect on cell growth/survival was examined. As control, cells were either mock-transfected or transfected with plasmid pCSP3, which expresses the full-length wild-type caspase-3 protein. It is noted that human embryonic kidney 293 cells have been shown to contain endogenous β-secretase activity, and have been widely used for studies of APP processing (Vassar et al., Science 286: 735-741 [1999]; Sinha et al., supra [1999]).

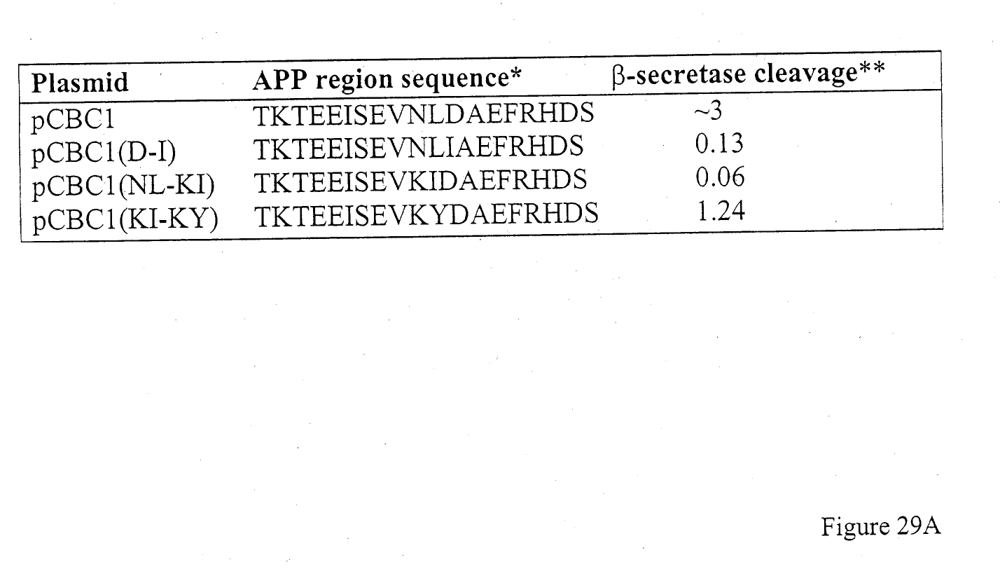

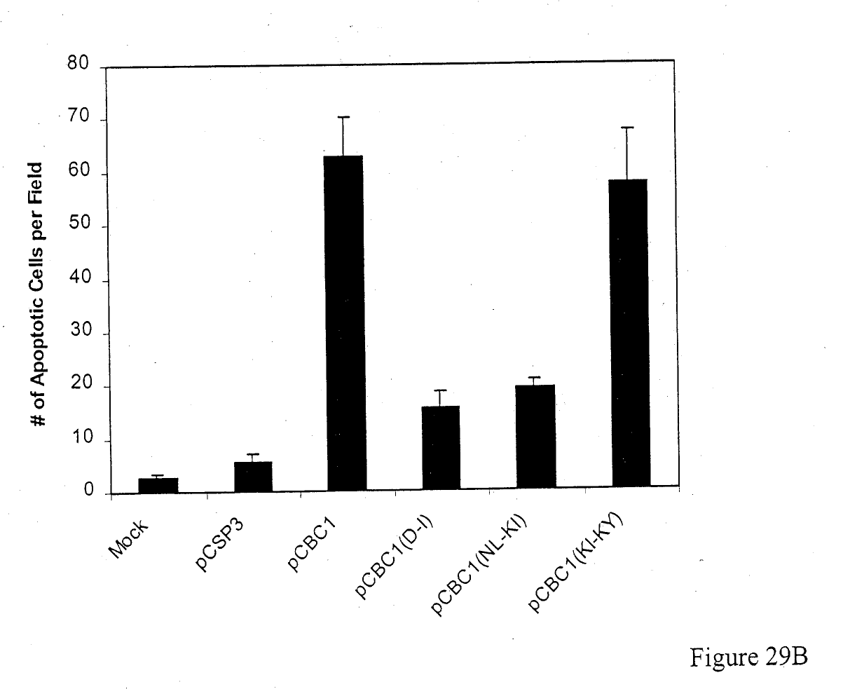

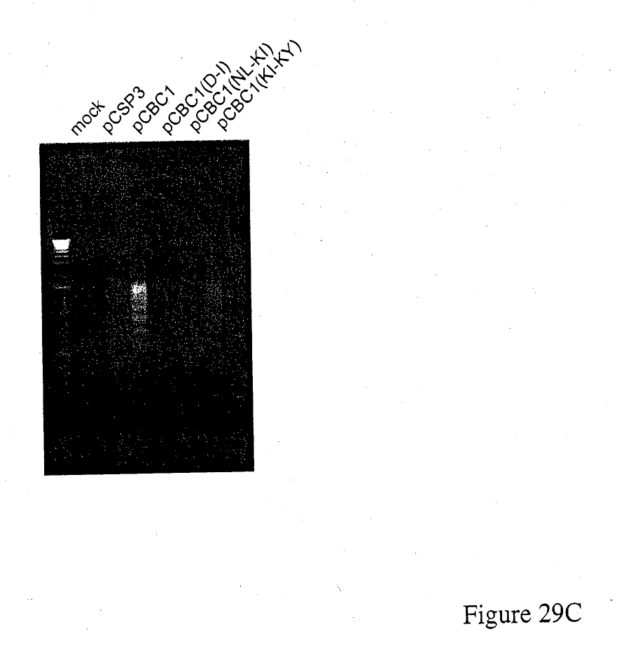

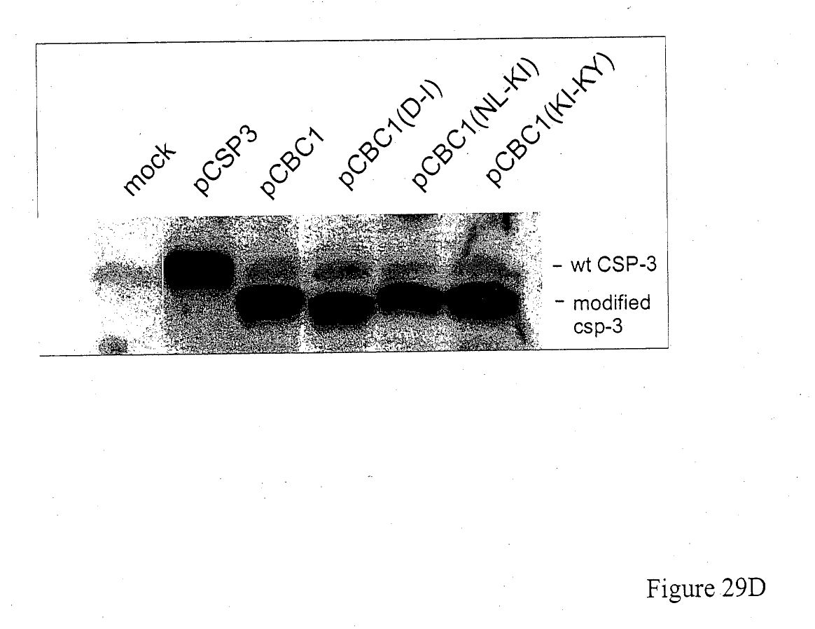

When the cells were examined under microscope 24 h after transfection, a large number of them showed signs of apoptosis in pCBC1-transfected dishes while there was virtually no or minimal cell death observed in mock or pCSP3-transfected dishes (FIG. 28B). The observed morphological features of apoptosis included cell blebbing, shrinking or rounding, and detachment from the dishes. In addition, nuclei fragmentation was also detected in pCBC1-transfected but not mock- or pCSP3-transfected cells using DNA ladder assay (FIG. 29C). Co-transfecting pCBC1 and a GFP-expressing plasmid and then monitoring apoptosis confirmed that the cell death was caused by transfection of pCBC1 plasmid. For example, most of the apoptotic cells were those transfected (green cells) and conversely majority of the transfected cells underwent apoptosis (FIGS. 28C and 28D).

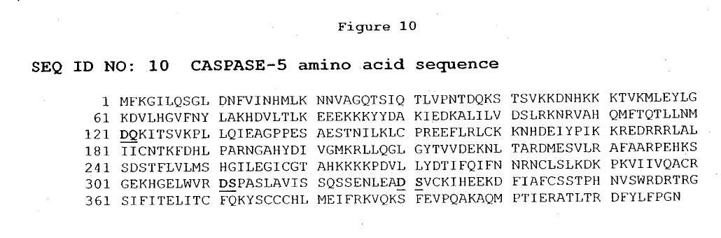

Further confirmation that the cell death was caused by caspase activity was obtained by treating cells with Z-VAD.fmk, a general caspase inhibitor, immediately after DNA transfection. As shown in FIGS. 28B, 28C and 28D, 10 μM Z-VAD.fmk significantly prevented cell death caused by transfection of pCBC1 plasmids, suggesting that the observed cell death was associated with caspase activity. The activation of caspase was monitored by measuring caspase-3 activity in transfected cells at various times after transfection using a caspase-3 fluorescent assay. More than 10-fold increase in caspase-3 activity was detected in pCBC1-transfected cells over mock-transfected cells 24 and 48 h after transfection (FIG. 28E). Taken together, these results indicated that the modified caspase-3 containing the β-secretase site induced apoptosis in 293T cells.

An indirect approach was used to determine whether the apoptosis caused by pCBC1 required β-secretase activity. For this, APP variants containing mutations surrounding the β-secretase cleavage site and having varying degree of susceptibility to β-secretase cleavage were used (Citron et al., Neuron 14: 661-670 [1995]) (FIG. 29A). It was reasoned that if β-secretase was involved in the modified caspase-3 induced cell death, then transfection of cells with variants of the modified caspase-3 containing mutations in the APP sequence should result in varying degree of cell death. Mutants of pCBC1 with alteration in the β-secretase recognition sequence were generated by site-directed mutagenesis. pCBC1(D-I) with D to I mutation (at P₁ site) (SEQ ID NO: 30) and pCBC1(NL-KI) with NL to KI mutation (at P₋₁ and P₋₂ sites) (SEQ ID NO: 31) were constructed (FIG. 29A). APP sequences containing these mutations are known to be less susceptible to β-secretase cleavage (Citron et al., Neuron 14: 661-670 [1995]). The extent of apoptosis was considerably reduced in 293T cells transfected with the mutant plasmids, pCBC1(D-I) and pCBC1(NL-KI), as determined by morphological examination (FIG. 29B) and DNA ladder assay (FIG. 29C). In order to further confirm the correlation of β-secretase activity with cell death, a third mutant was constructed by changing the amino acid I to Y (at P₋₁ site) in pCBC1(NL-KI) (SEQ ID NO: 32). The resultant construct was designated as pCBC1(KI-KY) (FIG. 29A). APP sequence containing KY was reported to be as good a substrate for β-secretase as the wild-type APP (Citron et al., supra [1995]). The values shown in FIG. 29A are in relation to the cleavage of the wild type sequence. Therefore, the wild type and the KY mutants are both about 3 times less efficient than the Swedish mutant site for cleavage (FIG. 29A). The extent of apoptosis observed in 293T cells transfected with pCBC1(KI-KY) was indeed similar to that in cells transfected with pCBC1 (FIG. 29B). The results obtained with the morphological assay, as described above, were corroborated in another assay that measured the induction of DNA ladder formation by various constructs upon transfection into cells (FIG. 29C). Specifically, the degree of DNA ladder formation was correlated with the susceptibility of the APP sequence in each modified caspase-3 plasmid (FIG. 29C).

Western blot analysis was used to examine the expression of caspase-3 in 293T cells transfected with the above plasmids. Compared with the endogenous caspase-3 in mock-transfected cells, pCSP3-transfected cells expressed significantly higher level of caspase-3 protein (FIG. 29D). Similarly, transfection of the various modified caspase-3 plasmids resulted in a comparable level of the modified caspase-3, which was detected as a 32 kD protein as expected (FIG. 29D). These and the above results suggested that the ability of pCBC1 to induce apoptosis is correlated with the requirement for secretase.

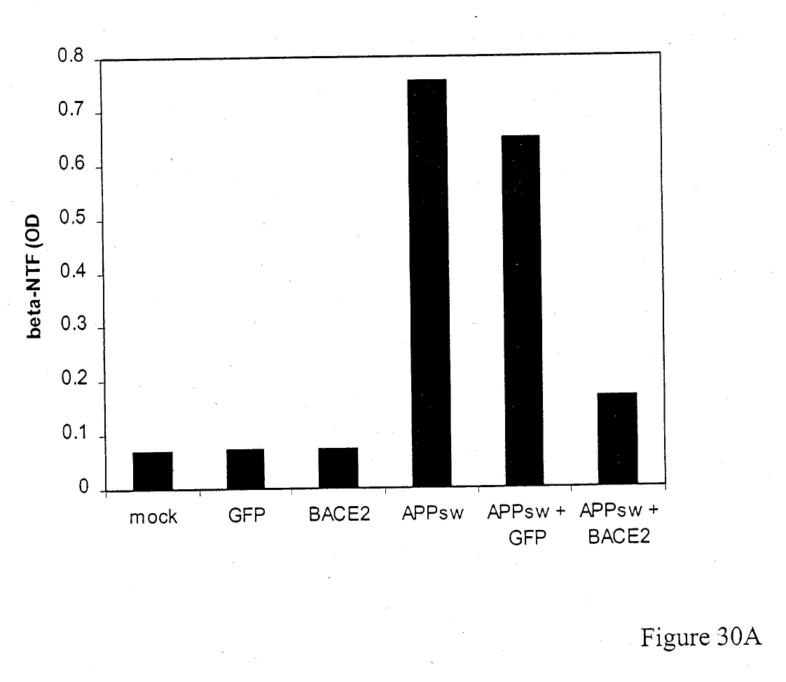

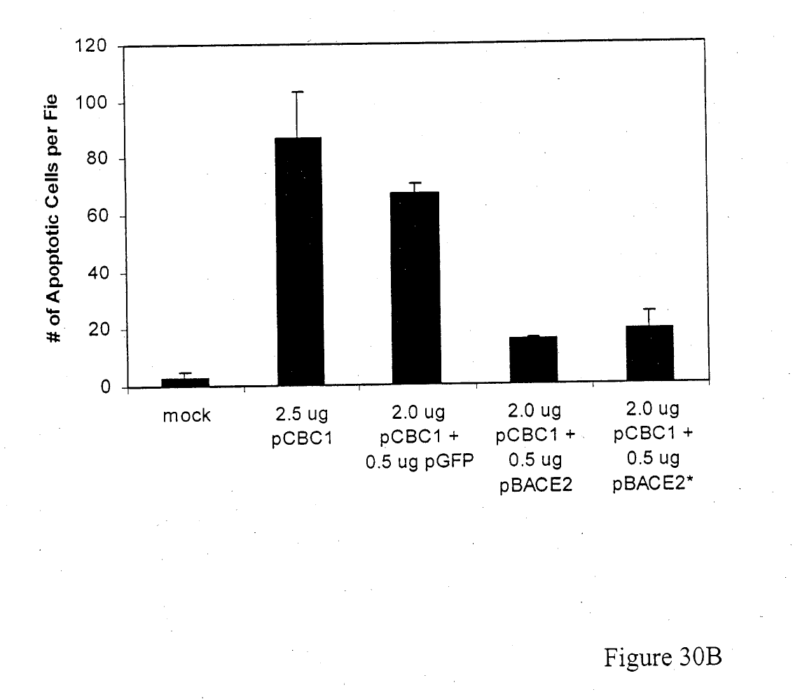

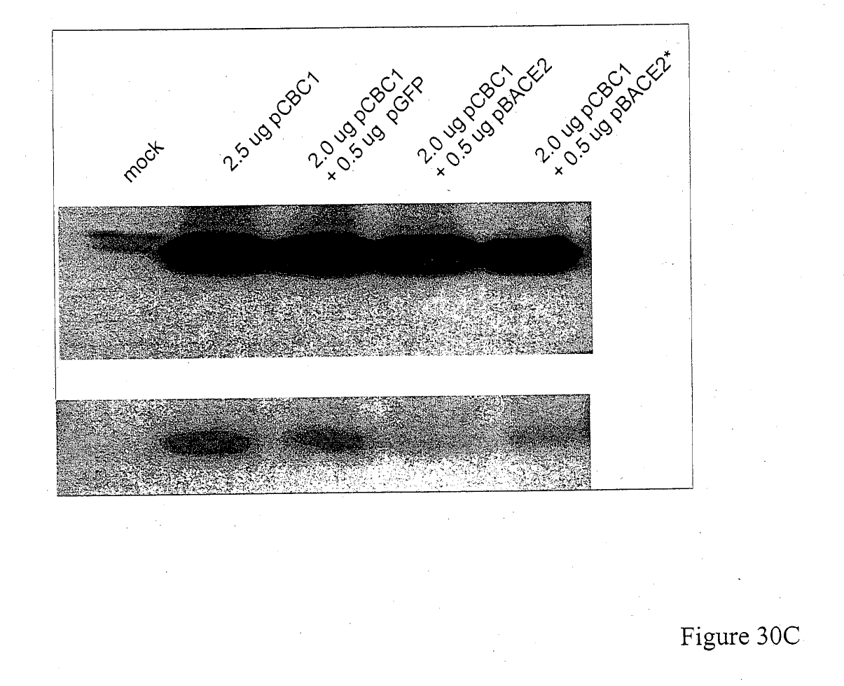

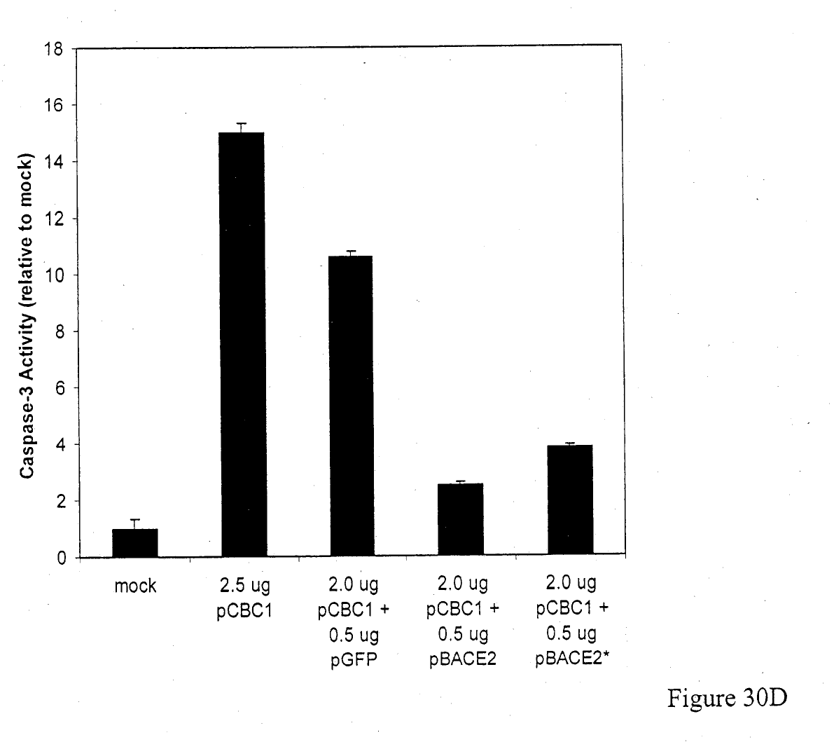

We have recently demonstrated that although BACE2 possesses weak secretase activity, it competes in vivo with β-secretase for β-site APP cleavage in an allosteric manner (Liu et al., submitted [2000]). The inhibition of the endogenous β-secretase activity by BACE2 in vivo was found to be independent of its catalytic activity since the catalytically inactive mutant of BACE2(D110A) also possessed similar inhibitory activity. As reported before (Liu et al., submitted [2000]) and shown in FIG. 30A, transfection of an APP expression plasmid into 293T cells resulted in increased release of the β-NTF product, which represents the N-terminal fragment from APP after β-secretase cleavage. However, co-transfection of BACE2 or BACE2(D110A) expression plasmid with the APP expression plasmid into 293T cells significantly inhibited the generation of β-NTF (FIG. 30A).

It was reasoned that if the modified caspase-3 is processed by the authentic β-secretase, then BACE2 or BACE2 mutant should also be capable of inhibiting the processing of the modified caspase-3 and the resultant cell death. The effect of BACE2 and BACE2 mutant on the processing and activation of the modified caspase-3 and on cell death caused by the modified caspase-3 was examined. As shown in FIG. 30B, co-transfection of a BACE2 expression plasmid with pCBC1 significantly reduced the number of apoptotic cells compared with controls. Co-transfection of the catalytically inactive BACE2(D110A) mutant with pCBC1 also resulted in a similar degree of inhibition on cell death caused by the modified caspase-3.

The processing of the modified caspase-3 and its activity in the absence or presence of BACE2 or mutant expression was examined. Cleavage of the modified caspase-3 at the β-secretase site should yield the large and small subunits of caspase-3, the latter of which was detected as a 12 kDa protein (FIG. 30C). The processed caspase-3 fragment was significantly reduced in cells co-transfected with pCBC1 and BACE2 or BACE2(D110A) plasmids compared with the control as shown in FIG. 30C. Similarly, co-transfection of BACE2 or mutant resulted in lower caspase-3 activity than the controls (FIG. 30D). The amount of the 12 kDa protein detected by Western blot (FIG. 30C) correlated with the caspase-3 activity (FIG. 30D), further supporting the identity of the 12 kDa protein as one of the subunits of the active caspase-3. Both BACE2 and BACE2 mutant plasmids gave rise to similar levels of BACE2 proteins (data not shown). Thus, BACE2 and BACE2(D110A) interfere with the processing of the modified caspase-3 in a manner similar to the APP processing at β-site, strongly confirming the involvement of β-secretase in the modified caspase-3 induced cell death.

All references cited throughout the present application, and the references cited therein, are hereby expressly incorporated by reference.

What is claimed is:

1. A method of identifying inhibitors of a protease comprising:
providing a eukaryotic cell susceptible to caspase-induced apoptosis, wherein the eukaryotic cell expresses a gene encoding a protease, and wherein the protease recognizes a proteolytic cleavage site;
introducing a fusion polypeptide expression vector into the eukaryotic cell, wherein the fusion polypeptide expression vector comprises an expression cassette encoding an inducible promoter that regulates a gene for a fusion polypeptide, wherein the fusion polypeptide comprises an amino acid sequence for a first caspase subunit, a linker sequence, a second caspase subunit, wherein the linker sequence comprises the proteolytic cleavage site;
introducing a cDNA expression vector into the eukaryotic cell, wherein the cDNA expression vector expresses an inhibitor that inhibits the protease from cleaving the proteolytic cleavage site of the fusion polypeptide in the eukaryotic cell;
inducing the expression of said fusion polypeptide;
isolating the eukaryotic cell after induction that contains the cDNA expression vector and survives induction; and
isolating the cDNA expression vector that expresses the inhibitor from the eukaryotic cell that survives induction.

2. The method of claim 1 wherein said first caspase subunit and second caspase subunit are the small subunit and large subunit subunits of a native human caspase-3 polypeptide.

3. The method of claim 1 wherein the caspase-induce apoptosis is induced by exogenous caspase-3 expression.

4. The method of claim 1 wherein said inducible promoter is a tet-operator modified CMV promoter.

5. The method of claim 1 wherein said protease is β-secretase.

6. The method of claim 5 wherein said eukaryotic cell is a MCF-7 cell.

7. The method of claim 1, wherein the gene encoding the protease is a gene exogenous to the eukaryotic cell.

8. The method of claim 1, wherein the gene encoding the protease is a gene endogenous to the eukaryotic cell.

9. The method of claim 1, wherein the inhibitor is a caspase inhibitor.

10. The method of claim 1 wherein said inhibitor is an antisense transcript of said protease.

11. The method of claim 1 wherein said inhibitor is a trans-dominant derivative of said protease.

12. The method of claim 1 wherein said inhibitor negatively regulates the activity of said protease.

13. The method of claim 1 wherein said inhibitor inhibits the expression of said protease.

14. The method of claim 1 wherein the cDNA expression vector is derived from a cDNA expression library.

15. The method of claim 14 wherein said library is a retroviral cDNA expression library.
